(19) United States
(12) Patent Application Publication
(10) Pub. No.: US 2024/0249839 A1
(43) Pub. Date: Jul. 25, 2024

(54) SYSTEMS, SOFTWARE, AND METHODS FOR MULTIOMIC SINGLE CELL CLASSIFICATION AND PREDICTION AND LONGITUDINAL TRAJECTORY ANALYSIS

(71) Applicant: lmmunai Inc., New York, NY (US)

(72) Inventors: Noam SOLOMON, New York, NY (US); Luis VOLOCH, New York, NY (US)

(21) Appl. No.: 18/101,102

(22) Filed: Jan. 24, 2023

(51) Int. Cl. G16H 50/20; G16B 20/00

(52) U.S. Cl. CPC G16H 50/20; G16B 20/00

(57) ABSTRACT

A system analyzes and maps the immune system through observational genomics, using multiomic single-cell technologies and machine learning, and uses functional genomics for therapy development including treatment outcome analysis, prediction, and recommendations based on multiomics and longitudinal trajectory analysis. Annotation is performed based on a multiomic classifier and the annotation is validated using RNA/protein pattern recognition per cell subset. Annotating may include automated cell type prediction including dimensionality reduction, feature extraction & automated cell type prediction, multiomic batch effect correction and multiomic based multiplet removal, and may involve separation of sub-cell types. Multiomic data used may comprise gene expression data, CITE seq markers, and TCR/BCR data. Cell type specific matching of clinical signatures with perturbation signatures may include mapping signature against large scale CRISPR perturbations in relevant cell type to identify potential drug targets. Mapping may include signature mapping to clinical covariate.

### CROSS-REFERENCE TO RELATED APPLICATIONS

### Field

This technology relates to methods of analyzing and mapping the immune system through observational genomics, using multiomic single-cell technologies and machine learning, and to functional genomics for therapy development including treatment outcome analysis, prediction, and recommendations based on multiomics and trajectory analysis. The technology herein further relates to systems, software, and methods based on a multiomic classifier that classifies different cell types each of which express many different genes, and for validating the classification, e.g., using RNA and/or protein pattern recognition per cell subset. The technology herein has applications in areas such as immunology, immunotherapy, immune system mapping, oncology, cell therapy, computational biology, and informed decision making for patient treatment and care.

### BACKGROUND

### The Immune System

The immune system is a complex network of cells and proteins that defends the body against disease. Immune cells are integral functional units of every organ in the body and have increasingly been recognized as key players in nearly every disease category, including cancer, cardiovascular disease, dementia, and cognitive dysfunction, as well as in other normal processes such as healthy aging. Because the immune system is implicated in nearly every disease, technology that analyzes the immune system to identify, diagnose, and treat disease, from cancer and infectious disease to autoimmune disorders, has the potential to transform the practice of medicine.

Yet, the immune system is amazingly complex. It is organized into many “families” of cells or cell types, each with distinct functions. Further, the immune system is capable of both innate and adaptive behaviors. The innate immune system is the body's first line of defense against germs entering the body. It distinguishes between things that are supposed to be inside the body, and pathogens and foreign substances that are not supposed to be in the body. It includes a “complement system” of complement proteins that, when activated along with macrophages, neutrophils and dendritic cells, can launch a cascaded defense to invaders. We are each born with an innate immune system, which has evolved over the course of many, many generations to protect our bodies from attack.

But just like any battle with enemy forces, the weapons and attack strategies of the pathogens attacking the body are constantly changing to defeat the body's defenses. The adaptive immune system is a bit like a library we are each born with, that has a particularized weapon or defense against every possible pathogen that has ever existed or may exist in the future. The adaptive immune system is able to specifically target a specific type of pathogen(s) by recognizing the pathogen(s) and then adaptively manufacturing specialized defense(s) to combat the specific pathogen(s). The adaptive immune system has the advantage of being able to “remember” pathogens the body has been exposed to in the past, so the next time a known pathogen is encountered, the adaptive immune system can respond faster. Vaccines work by invoking such adaptive immune responses.

The adaptive immune system uses cytokine/chemokine signaling to modulate the immune response, including by recruiting additional immune cells including specialized cells (e.g., B and T cells or lymphocytes) and proteins (e.g., antibodies) that detect and eliminate specific pathogens. Some immune system cells or cell types are inflammatory while others are immunosuppressive, e.g., T-cells including the CD8, CD4, and regulatory T-cells (these are different kinds of lymphocytes—a type of white blood cell), B-cells in the adaptive immune system, macrophages (e.g., M1 and M2), dendritic cells, and other cell types of the innate immune system, each having different transcriptional and epigenetic profiles. To further complicate matters, each immune cell type may display a complex combination of attributes based on that cell type's unique pattern of expression of thousands of genes. See e.g., Delves et al, “The Immune System”, Jul. 6, 2000, N Engl J Med 2000; 343:37-49 DOI: 10.1056/NEJM200007063430107; Spiering et al, Primer on the Immune System, Alcohol Res. 2015; 37(2): 171-175; Sompayrac, “How the Immune System Works”, 6^{th} Ed. (John Wiley & Sons 2019); Rich et al, Clinical Immunology Principles and Practice, 6^{th} Ed, (Elsevier 2022); https://www.ncbi.nlm.nih.gov/books/NBK279396/; Dettmer, “Immune: A Journey into the Mysterious System That Keeps You Alive” (Random House 2021).

The nature of an individual's immune response to a particular disease thus depends on the composition of and interactions between their immune system cells. The ways these various cells interact with one another is also quite complex. Typically hundreds of different cell types, cell populations, and other factors may be associated with any given set of immune response(s). These immune cell types are often not totally distinct in each tissue and disease. Rather, the same or similar cell types may play similar roles across different tissues and diseases. For example, the set of immune cell types fighting a melanoma may be the same cell types combatting a viral infection and may also be the same cell types implicated in the onset of autoimmune disease. The immune cell types present in the melanoma may be the same cell types present in the cardiovascular system, brain, intestinal tract, or lungs. Therefore, knowledge of the immune building blocks learned in one organ or disease setting may be transferable to the context of additional organs, tissues, and/or diseases. Once understood, these features may be generalizable to a wide variety of disease settings for diagnostic, disease monitoring, and therapeutic purposes.

### Use of RNA Sequencing

A worthwhile goal may be to obtain a “snapshot” of the state of a person's immune system for example by characterizing expressed immune system cell types, their populations, and use RNAseq to provide information on expressed cell types by cell-type specific cell surface markers. In particular, a traditional basis of understanding features of the diverse components of the immune system that are common to many organ systems and tissues is to use high-resolution genomic technologies such as single-cell RNA-sequencing (scRNA-seq). RNA sequencing (“RNA-seq”) is a transcriptomic approach for the detection and quantitative analysis of messenger RNA molecules in a biological sample. RNA-seq is thus useful for studying cellular responses. See e.g., Haque, “A practical guide to single-cell RNA-sequencing for biomedical research and clinical applications,” Genome Med 9, 75 (2017), https://doi.org/10.1186/s13073-017-0467-4; Hwang et al., “Single-cell RNA sequencing technologies and bioinformatics pipelines”, Exp Mol Med 50, 1-14 (2018), https://doi.org/10.1038/s12276-018-0071-8. To perform scRNA-seq, a sample is prepared and a single cell library is constructed. Sequencing and a bioinformatics analysis are then performed. The resulting transcriptional profiling of many individual cells can reveal complex and rare cell populations, uncover regulatory relationships between genes, and track the trajectories of distinct cell lineages in development. See Hwang et al. For example, scRNA-seq permits comparison of the transcriptomes of individual cells (e.g., to identify rare cell populations that would otherwise go undetected in analyses of pooled cells) and can also provide important information about fundamental characteristics of gene expression. See e.g., Haque.

Each one of the estimated 30 trillion cells of a human is unique at a transcriptional level. Performing bulk or whole-tissue RNA sequencing, which combines the contents of millions of cells, masks most of the differences between cells as the resulting data comprises of the averaged signal from all cells. Single-cell RNA-sequencing (scRNA-seq) has emerged as a revolutionary technique, which can be used to identify the unique transcriptomic profile of each cell. Using this information, it is possible to identify of new cell types, resolve cellular dynamics of developmental processes, and identify gene regulatory mechanisms that vary between cell subtypes. Cell type identification and discovery of subtypes has thus emerged as one of the most important early applications of scRNA-seq. Prior to the arrival of scRNA-seq, the traditional methods to classify cells were based on microscopy, histology, and pathological criteria. In the field of immunology, cell surface markers have been widely used to distinguish cell subtypes, for a wide range of purposes. While this approach is desirable in practical terms for cell isolation, e.g., via fluorescence-activated cell sorting (FACS), these markers may not reflect the overall heterogeneity at a transcriptomic and phenotypic level from mixed cell populations. See FIG. 2A. Unsupervised and supervised clustering approaches have been used to determine groups of cells based on similar transcriptional signatures within a sample, and frequently, cells within a cluster are collectively labeled based on the average expression levels of canonical markers. The cluster-based classification methods assume that all cells within a cluster are the same type and thus can be labeled collectively. This assumption is not always right, with clusters sometimes containing small percentages of multiple cell types in addition to a major cell type. A method that classifies each cell individually, without clustering first, solves these problems and should provide higher overall accuracy in cell labeling. See e.g., Alquicira-Hernandez et al, “scPred: accurate supervised method for cell-type classification from single-cell RNA-seq data”, Genome Biology volume 20, Article number: 264 (2019).

To be able to predict the classification of a single cell based upon its transcriptome read-out, first, a prediction model needs to be built where the effects of given features are estimated. It is clear that both the selection of features and estimation of their effects play a critical role in the overall prediction performance. Unlike prediction methods that use data derived from bulk RNA-seq data where gene expression averages are used as features, phenotype prediction at single-cell level faces new challenges. Firstly, cell-to-cell differences must be considered to define and predict cell types. Using only a subset of genes (e.g., differentially expressed genes) will likely exclude discriminant sources of variation across cells. An additional limitation is the inconsistency seen between statistical methods used to identify differentially expressed genes. Also, if the number of observations that define a specific subtype of cells is high, then classification algorithms can be computationally expensive or suffer from overfitting. Id.

There are numerous applications for which prediction of a cell state or type from its scRNA-seq data can play an important role. An obvious example is in the burgeoning use of single-cell data in characterizing disease states and underlying biology at single-cell resolution. The granular nature of single-cell characterization has enormous implications for the accurate prediction of specific cell subtypes and pathological-related states. Such prediction strategies will play an important role in the early diagnosis of diseases or informing personalized treatment. Similarly, efforts arising from the Human Cell Atlas are set to create a comprehensive reference atlas of most cell subtypes in the human body, meaning cells from new samples can be mapped against this reference. Id.

### The Multidimensional Complexity of the Immune System

Despite the power of such techniques, complexities of the immune system often result in vast amounts of potential data to be analyzed containing many disparate dimensions, for example:
many thousands of gene expression values
information about the B cell repertoire
repertoire/expression levels for B cell receptors (BCRs)/clonotypes (which may also be reflected in the repertoire of soluble antibodies)
information about the T cell repertoire (e.g., TCR clonotypes)
expression levels for antibodies (which are proteins, as are TCRs, and BCRs)
information about a range of other proteins (e.g., cytokines, etc.).

Note: the input data set may or may not contain both “soluble antibodies” and “BCR receptor” data. Typically, there should be a reasonable correlation between BCR data and the profile of soluble antibodies present in a patient sample, but they shouldn't be identical—as just one example, one might capture memory B cells induced by vaccination or prior pathogen exposure by looking at BCR data that one might not see if just looking at the profile of soluble antibodies present in a sample.

This high dimensional data provides unique computational and other challenges for analysis, such as:
i) how to analyze so many dimensions,
ii) how to account for batch effects (see https://genomicsclass.github.io/book/pages/intro_to_batch_effects.html) (e.g., how to identify whether information might be compromised by a batch effect, how to determine what impact the batch effect has had on the information, and how to correct or compensate for the batch effect. In a general case, one might not know a priori that a particular dataset has been impacted by a batch effect, so the challenge is not only trying to figure out how to spot information that might be compromised by a batch effect, but also how to determine what impact the batch effect has had on that information, and how to correct for that.)
iii) how to annotate information about these cells and their biological functions in an automated way.

However, because the immune system is so complex, there are thus challenges to obtaining an accurate “snapshot” of such immune state information. Because of such challenges, single-cell RNA sequencing by itself has in the past been limited in its ability to provide insights into how to manage the clinical progression of disease of a particular patient, or into transitions between immune states as a person undergoes clinical treatments for their disease (or diseases, in the case of patients having co-morbidities). Because of these and other challenges, existing techniques are often unable to distinguish different cell types with sufficient precision to enable accurate identification of the cell types present, resulting in the situation shown in FIG. 2A. Different cell types may not be fully distinguished, and rare cell types may be missed altogether.

It is possible to obtain (additional) information about the immune system using Cellular Indexing of Transcriptomes and Epitopes by Sequencing (CITE-seq) (https://cite-seq.com/), but that method doesn't provide a comprehensive look at the surface proteome, just a subset of surface proteins. It is also possible to get some information about cell types from scRNAseq—like figuring out whether there are CD4+ or CD8+ T cells, etc. A sequence of such immune snapshots could theoretically represent a progression of immune states of that person—indicating for example progression of a disease or a recovery. But turning this from theory into practice requires a substantial amount of innovation.

### Use of Clinical Trajectory Analysis

A “longitudinal” study refers to an investigation where participant outcomes and possible treatments or exposures are collected at multiple follow-up times. Such repeated measures of data are typically correlated within subjects and thus require special statistical techniques for valid analysis and inference. Longitudinal studies can thus be used to characterize normal growth and aging, to assess the effect of risk factors on human health, and to evaluate the effectiveness of treatments. See e.g., https://faculty.washington.edu/heagerty/Courses/VA-longitudinal/private/LDAchapter.pdf.

A “trajectory” describes the course of a measured variable over age or time. Clinical trajectory analysis is a potential tool that has been used in the past to help predict and determine clinical outcomes. Clinical trajectory analysis is a technique where the medical records (for example, as recorded in electronic health records (EHRs) of a person or a group of people) are analyzed to identify clinical diagnosis and treatments prescribed, and to determine clinical outcomes of those treatments. See e.g., Giannoula et al, “A system-level analysis of patient disease trajectories based on clinical, phenotypic and molecular similarities”, Bioinformatics, Volume 37, Issue 10, 15 May 2021, Pages 1435-1443; Allam et al, Analyzing Patient Trajectories With Artificial Intelligence, J Med Internet Res 2021; 23(12):e29812 doi: 10.2196/29812; Kehl et al, “Artificial intelligence-aided clinical annotation of a large multi-cancer genomic dataset,” Nature Communications volume 12, Article number: 7304 (2021). But how to effectively employ longitudinal trajectory analyses in a scalable way using the high data dimensionality of the immune system has been a challenge. See e.g., Zheng et al, “Immune cell and TCR/BCR repertoire profiling in systemic lupus erythematosus patients by single-cell sequencing”, Aging Vol. 13 No. 21 (2021).

Artificial intelligence (“AI”) techniques have been used in conjunction with clinical trajectory analysis to attempt to identify subpopulations that are likely to respond to a treatment based upon demographic and medical records. See e.g., Harrer et al, “Artificial Intelligence for Clinical Trial Design”, Trends in Pharmacological Sciences Volume 40, Issue 8, August 2019, Pages 577-591 https://doi.org/10.1016/j.tips.2019.05.005; Brasil et al, “Artificial Intelligence (AI) in Rare Diseases: Is the Future Brighter?,” Genes (Basel) 2019 December; 10(12): 978, Published online 2019 Nov. 27. doi: 10.3390/genes10120978. These systems have had success in identifying successful and unsuccessful treatments based upon patient demographics (e.g., membership in an ethnic subgroup). However, many such previous attempts measured only broad responses across a population and did not take into account each individual's genome and current state of their immune system—meaning that personalized medicine was not achieved. In addition, many of these data inputs often require manual correction and/or tagging to enable the AI techniques, which makes the techniques costly and difficult to scale to large patient populations. See e.g., Ghassemi et al, “A Review of Challenges and Opportunities in Machine Learning for Health”, AMIA Jt Summits Transl Sci Proc. 2020; 2020: 191-200, Published online 2020 May 30.

In addition, integrating a patient's medical records (e.g., from a source such as an EHR) with genomic and multiomic information, and immune state requires complex and constantly evolving methods. The task is sufficiently computationally complex that the integration of these different information sources to yield a probability weighted prognosis is often not currently computationally feasible for many patient populations.

### Challenges Ahead

Thus, while substantial work has gone into addressing the problems discussed above, existing solutions generally do not scale to addressing hundreds of samples or millions of cells. Because of the information complexity, what is needed are data analysis and computational tools that can distill the high-dimensional data into meaningful, clinically relevant information. For example, it would be highly useful to be able to isolate—in an automated, scalable way—at least one distinct subset of a plurality of different immune system single cell populations that other analysis indicates are exhibiting evolving molecular changes.

What is also needed is an efficient system that performs such computations automatically at scale in computationally feasible ways, and which produces reliable, reproducible probability weighted visualizations and prognoses for a patient or patient group based on a combination of multiomic data, medical records, and one or more immune states across co-morbid/multiple disease conditions.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Non-limiting features of exemplary embodiments will best be understood from the below detailed description of exemplary embodiments shown in the accompanying drawings, of which:

FIG. 1 shows an example process.

FIG. 1A shows a more detailed example process.

FIG. 2A depicts an exemplary prior art cell type group clustering.

FIG. 2B depicts an exemplary improved machine learning cell type distinguished grouping produced using the described process.

FIG. 3 shows a prior art annotation process.

FIG. 4A is an exemplary heatmap of T cell marker genes.

FIG. 4B is an exemplary heatmap of T cell marker proteins.

FIG. 5 is an exemplary flowchart of cell type specific matching of clinical signatures with perturbation signatures.

FIGS. 5A, 5A-1, 5A-2, 5A-3 are together an exemplary more detailed flowchart of a refined signature of a-PD1 response at the cellular level.

FIG. 6A is a flowchart of an exemplary non-limiting platform use case of clinical covariate enrichment.

FIG. 6B shows exemplary graphs of signature mapping to clinical covariate (response to a-PD1).

FIG. 7A is a graph showing exemplary mapping perturbations to PD1 response signature.

FIG. 7B shows the FIG. 6B exemplary signature mapping to clinical covariate (response to a-PD1) with DHX37 identified based on the mapping perturbations.

FIG. 8 is a flowchart of an exemplary longitudinal trajectory analysis.

FIG. 9 shows exemplary graphs of how T cell clonal dynamics associate with response.

FIG. 10 shows an exemplary graph of CD8 TEMRA cells specifically peaking in a response-associated trajectory.

FIG. 11 shows exemplary trajectory enrichment between treatment arms.

FIG. 12A shows exemplary histograms of baseline exhausted clones collapsing and fresh clones tending to expand.

FIG. 12B shows exemplary graphs of baseline exhausted clones collapsing and fresh clones tending to expand.

FIG. 13 shows a flowchart of exemplary association of cell specific molecular phenotypes with clinical covariate response.

FIG. 14 shows exemplary exhaustion signals in CD8 non-naïve increasing in responders post treatment, with exhaustion signature scores higher in responders post treatment.

FIG. 15 is a schematic diagram of an exemplary predictor generation system (“PGS”) including a probabilistic predictive network graph generator system (“PPNGGS”) and a patient outcome predictive system (“POPS”).

FIG. 16 is a schematic block diagram of an exemplary computing apparatus supporting aspects of the probabilistic predictive network graph generator system (PPNGGS).

FIG. 17 shows an exemplary configured VAE or other multi-level machine learning component such as a neural network, illustrating inputs, VAE encoder/decoder-generator portions, and resulting reduced dimensional outputs.

FIG. 18 is a schematic block diagram of an exemplary computing apparatus supporting aspects of a patient outcome predictive system (POPS).

FIG. 19 is a schematic block diagram of an illustrative exemplary computer server that supports the described system(s) shown in FIG. 18.

FIG. 20A is an exemplary trajectory diagram depicting a starting profile cluster of multiple patients and intermediate states for particular patients that lead to different target cluster states.

FIG. 20B is an exemplary diagram depicting a machine learning algorithm's detection of when a trajectory of states for a particular patient begins to trend outside of the graph variance.

FIG. 20C is an exemplary diagram depicting a machine learning algorithm's prediction of different endpoint states that each depend from similar intermediate states.

### DESCRIPTION OF EXEMPLARY EMBODIMENTS

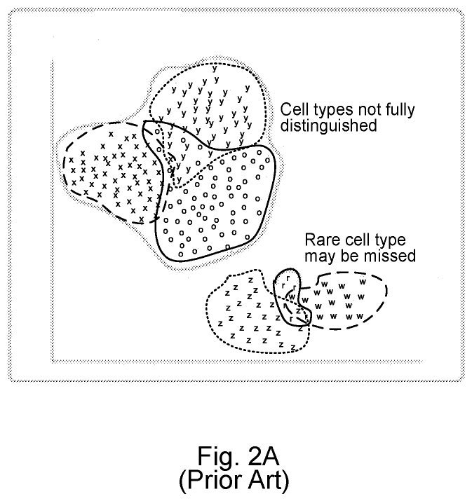

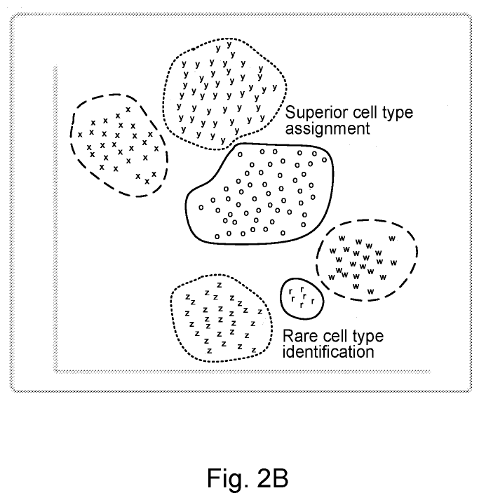

The present specification describes techniques for automatically deriving multiomic signatures and relating those multiomic signatures to clinical outcomes. The exemplary non-limiting technology in one embodiment uses at least one identified cell type (e.g., annotated in a multiomic way) which is relevant for predicting clinical outcomes. Using particular multiomic input data informs trajectories or node selections e.g., on a graph or other representation, to provide results that are qualitatively different from what can be determined using different and/or fewer input data types and different analytical techniques. FIG. 2B shows the type of improved results that may be obtained, including but not limited to superior cell type alignment and rare cell type identification.

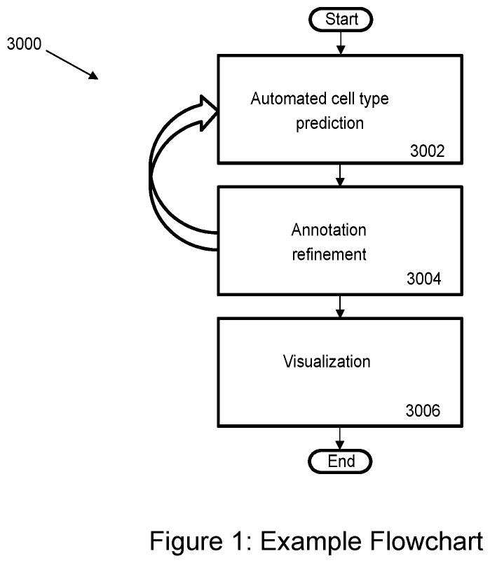

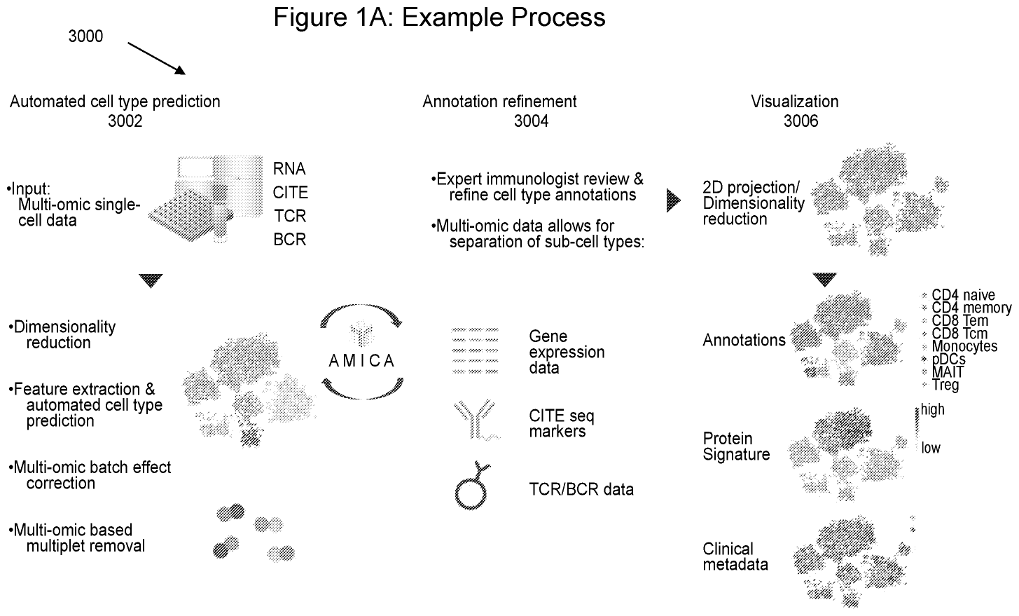

FIG. 1 shows an example flowchart of an automated process 3000 that includes three major steps, operations or phases:
Automated cell type prediction 3002
Annotation refinement 3004
Visualization 3006.

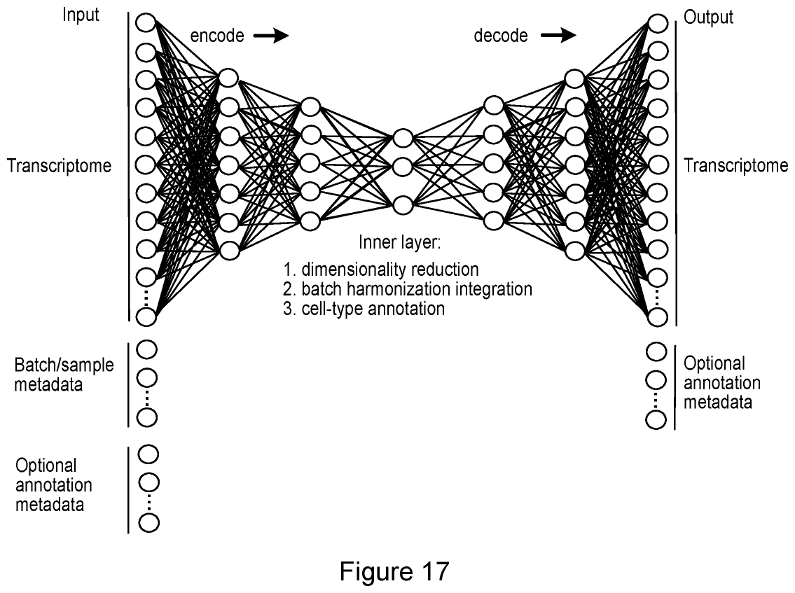

The process 3000 shown is a multi-step process which includes machine learning (“ML”) models for automated cell type prediction and classification using multiomic data as input. The ML models may be implemented by one or more computer processors connected to one or more non-transitory memories storing instructions that are executed by the computer processors. For example, such ML models may comprise or constitute neural networks such as deep neural networks (see FIG. 17 for one example such structure), convolutional neural networks, autoencoders and associated architectures, or other implementations, that are implemented or performed by CPUs and/or GPUs including hardware-based tensor processing units. Such neural networks are trained using test data sets to develop neural network level coefficients that are then used to recognize patterns in multiomic input data to provide more reliable cell type prediction, annotation refinement and/or visualization. More detailed discussion of such implementations is provided below.

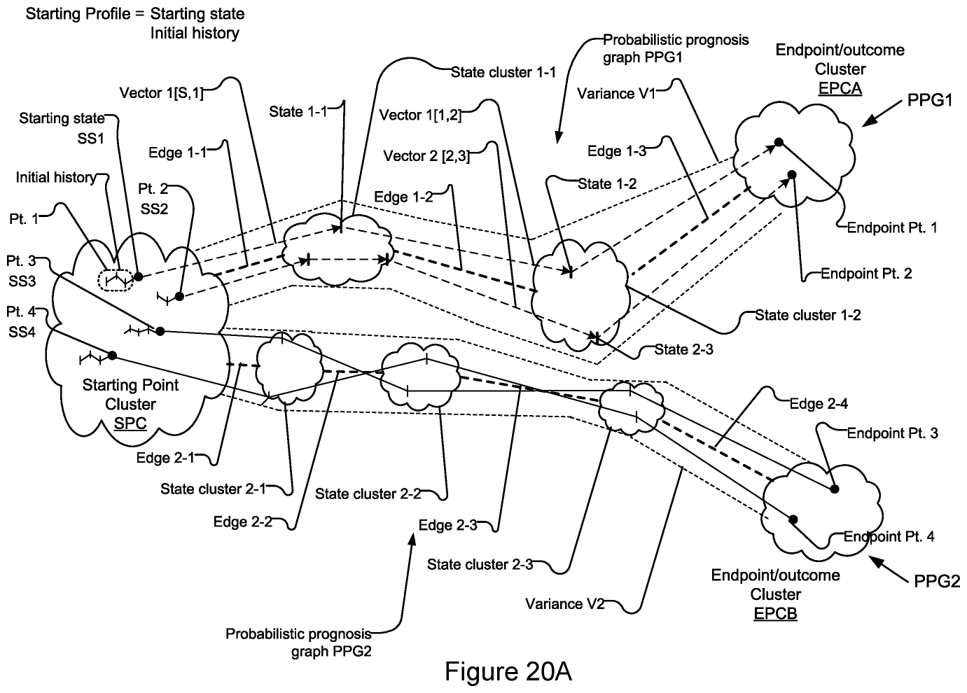

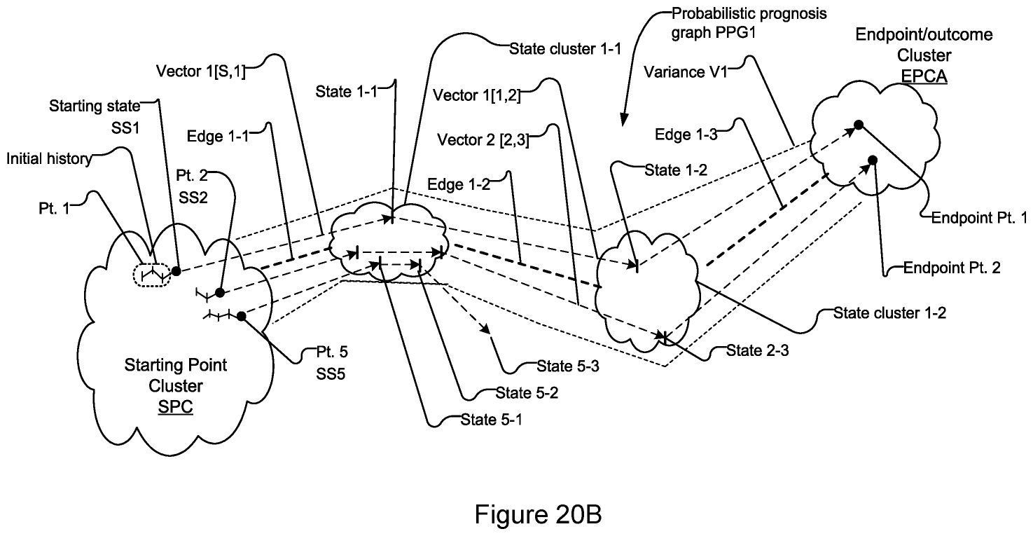

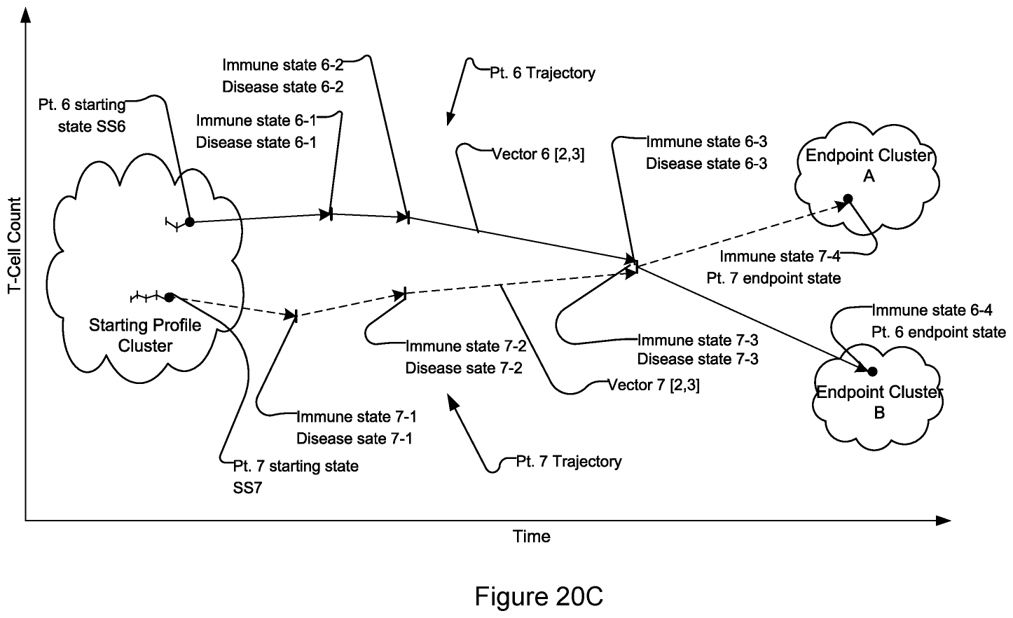

As shown in the FIG. 1 flowchart, the different operations while discrete can iterate and interact with one another. For example, annotation refinement 3004 may operate on annotation results from automated cell type prediction 3002, and the results of annotation refinement 3004 may be fed back to automated cell type prediction 3002 for further processing and analysis until a desired precision and/or data depth is obtained. Similarly, visualization 3006 may provide results that can be used to improve or change automated cell type prediction 3002 and/or annotation refinement 3004. Furthermore, visualization 3006 as described herein may include for example more extensive analysis such as producing graphs based on clinical covariates, such as shown in FIGS. 20A, 20B, 20C. Thus, the result of the process could sometimes be a display on a computer screen, but may also be a data structure or representation that can be further analyzed to predict disease outcome in a group of patients or a single patient (personalized medicine).

### Multiomics Data Input

In the FIGS. 1 and 1A example shown, data inputted to the automated cell type prediction ML model(s) 3002 includes multiomic single cell data, namely RNA, CITE, TCR and BCR. Thus, in one embodiment at least four different single cell data types are used as input for the predicting and annotating single cells, for example:
scRNAseq
CITE-seq (Cellular Indexing of Transcriptomes and Epitopes by Sequencing—a method for performing RNA sequencing along with gaining quantitative and qualitative information on a small number of surface proteins with available antibodies on a single cell level, e.g., by looking at a subset of expressed cell surface proteins and thus a subset of surface protein expression)
TCR (T cell receptor) profiling
BCR (B cell receptor) profiling.

In this context, “single cell” does not necessarily mean the automated call type prediction 3002 operates on only one cell type. Rather, in some modes, the prediction 3002 may predict a plurality or number of different single cell types. See FIG. 2B.

In one embodiment, the automated cell type prediction process 3002 performs, based on multiomic input data:
a dimensionality reduction,
extracting features and performing automated cell type prediction,
perform a multiomic batch effect correction (to remove variability from the data that is not due to the variable(s) of interest); and
performs multiomic based multiplet removal.

As explained below, a result or output of the automated cell type prediction 3002 in one embodiment may be or comprise labels and a transcriptome annotation that represents cell type prediction based on a dataset representing a significant population of cells of a subject or group of subjects. Such datasets may be derived using conventional lab testing techniques that harvest cells from a human subject or a group of human subjects.

One embodiment performs these block 3002 processes and in particular the feature extraction and automated cell type prediction using an artificial intelligence (AI) multiomic classifier providing multi-level processing which includes one or more machine learning (ML) models for automated cell type prediction and which is/are capable of using multiple dimensions of multiomic data as input.

In one embodiment, classification and prediction is performed automatically based on at least transcriptomic data and surface proteomic data, plus potentially limited (e.g., TCR/BCR only) genomic sequencing data. The dimensionality reduction shown is used to reduce the number of dimensions the classifier must operate on.

TCR profiling and BCR profiling each read different parts of the cell specific mRNA than the scRNA protocol. Neither assay reads all RNA sequences from the cell. Instead they each generally select a subset that includes the relevant information. For general scRNA protocol, the goal usually is to read lots of unique short sequences from the end of RNA molecules to create a catalogue of what is expressed. For TCR/BCR analysis, the goal is usually to read the full TCR/BCR message to know how they differ cell to cell. The example embodiment uses machine learning to integrate these different goals and associated results, and provide enhanced predictive results that none of the underlying individual technique can reliably provide on its own.

The TCR/BCR profiling may be done on genomic DNA. It is also possible to look at TCR and BCR expression when performing scRNAseq. There may be overlap between the scRNAseq data and the TCR/BCR data, but there may also be information obtained from the TCR/BCR profiling that is not obtained from the scRNAseq analysis because the scRNAseq data and the TCR/BCR data are different. For example, if one uses a 10× Genomics Chromium instrument, the cDNA obtained from each single cell to be analyzed will be split into two aliquots, one used to make a library for T/BCR profiling, and one for RNAseq (see, e.g., https://www.10xgenomics.com/products/single-cell-immune-profiling).

Batch effects in omics datasets are usually a source of technical noise that masks the biological signal and hampers data analysis. Batch effect removal has been widely addressed for individual omics technologies. However, multiomic datasets may combine data obtained in different batches where omics type and batch are often confounded. Moreover, systematic biases may be introduced without notice during data acquisition, which creates a hidden batch effect. It is desirable to perform batch effect correction to remove batch effect in the multiomics data sets. See for example Ugidos et al, “MultiBaC: an R package to remove batch effects in multiomic experiments”, Bioinformatics, Volume 38, Issue 9, Pages 2657-2658, doi.org/10.1093/bioinformatics/btac132 (1 May 2022).

Multiplets can create artificial cell types in the dataset. In one example context, multiplet is when more than one cell, e.g., 2 cells are processed as if they were a single cell. The phenotype of a multiplet is the sum of two cells. The problem is how to distinguish a multiplet from a single cell with a new phenotype. See, e.g., Xin et al, “GMM-Demux: sample demultiplexing, multiplet detection, experiment planning, and novel cell-type verification in single cell sequencing”, Genome Biol 21, 188 (2020). https://doi.org/10.1186/s13059-020-02084-2. Identifying and removing multiplets for example can improve the scalability and the reliability of single cell RNA sequencing (scRNA-seq). In the FIG. 1A example, the multiplet removal process is not limited to scRNA-seq data, but instead extends to the other omics and associated datasets analyzed as well.

The automated cell type prediction 3002 in one embodiment may produce what is known as an annotation or transcriptome annotation, e.g., by “marking” (in an automated way) additional information or in some cases corrections on labels of cell patterns to indicate predicted cell types and/or functions. In one embodiment, such annotation comprises updating, with at least one processor, one or more data structures that store identifications of observed cell populations and/or characteristics thereof. In one embodiment, annotation 3002 may be performed in accordance with US20210057107.

By way of further background, annotation is usually thought of as a prediction of a function of a gene or DNA region of a chromosome, using computational means. See e.g., Koonin et al, “Sequence—Evolution—Function: Computational Approaches in Comparative Genomics” (Kluwer Academic 2003), in particular Chapter 5 (“Genome Annotation and Analysis”). The “unit” of genome annotation is typically the description of an individual gene and its protein (or RNA) product, and the focal point of each such record is the function(s) assigned to the gene product. Annotation may involve reviewing data that represents a snapshot of a cell state and “marking it up” for the benefit of subsequent users. Software is typically used to run routine tasks in a batch mode and also to organize the results from different programs in a convenient form (each genome project may employ one or another set of tools to achieve this). In previous types, annotation was often performed by an immunologist literally marking a printout of a transcriptome with a pen or pencil. However, in example embodiments herein, at least one processor is used to automatically revise, edit and/or augment a data structure stored by a computing system.

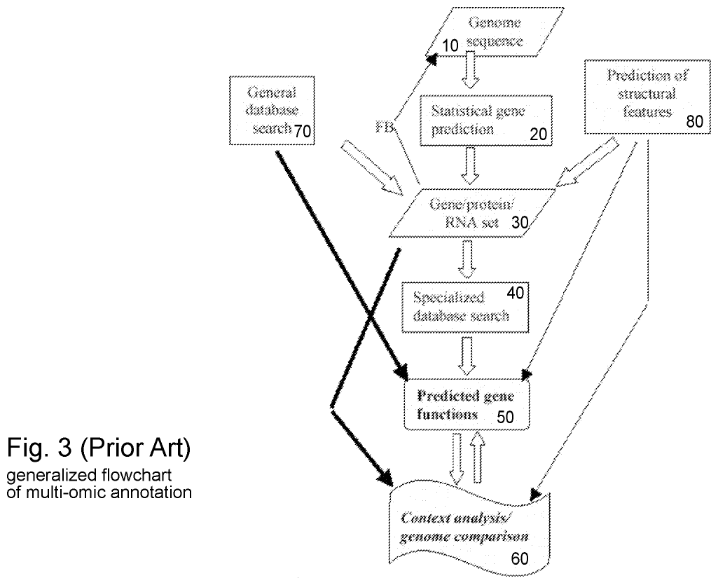

FIG. 3 shows a prior art generalized flowchart of an exemplary annotation process for a DNA region of a gene. In the approach shown, the gene is sequenced (10) and statistical based processing is performed (20) to predict protein coding genes in the sequence. Feedback (FB) can be used to correct sequencing errors (e.g., frameshifts) in the DNA sequence. General sequence databases (70) are then searched for homology (similar sequences). Next, predictions of structural features are made (80). Such structural feature prediction can, for example, identify signal peptide sequences, transmembrane sequences or segments, coiled-coil domains, and other features of putative protein structure that can impact protein function. Specialized databases are typically searched (40) for conserved domains, identification of orthologous relationships and refined functional prediction, metabolic pathway reconstruction, and possibly, other information. Then, genome context analysis and functional predictions are made (50). Homology analysis (looking for similar sequences that have already been identified) is a common technique used in such functional predictions. Context analysis/genome comparison (6) is then often performed. As discussed above, example embodiments may perform such annotation using multiomics to provide better predictions than can be obtained from single omics. See US20210057107.

While the multiomic annotation approach used by some embodiments may provide more precise and accurate prediction of single cell type than other, single omic techniques, embodiments herein further enhance the process by performing multiomic annotation refinement 3004 to attempt to refine the multiomic annotation developed by step 3002. In example embodiments, human experts are supported by automated algorithms on multiomic data to guide the annotation refinement process. In another embodiment, the process is largely or entirely automatic and performed for example by a computer architecture described below. Thus, step 3004 in some examples can include expert (human and/or expert system) immunologist review to refine cell type annotations, and/or automated processes may be used instead or in addition and/or in conjunction.

In performing this process, multiomic data allows for separation of sub-cell types, for example:
gene expression data,
CITE-seq markers, and
TCR/BCR data.

To further enhance results, an Annotated Multiomic Immune Cell Atlas (AMICA™) database may be used—and in some cases modified through iteration of the automated cell type prediction 2002 and annotation refinement 3004. Such a database may be used for example to perform a multiomic homology analysis. As mentioned above, prediction 3002 and refinement 3004 may be performed iteratively until a desired precision or data depth is reached. All of these processes in one embodiment are also informed by the AMICA™ database in an iterative process where the automated and/or manual annotation may happen one after another or in parallel.

Once the annotation has been refined (3004), the results can be displayed or otherwise presented for visualization purposes (3006), e.g., for context. Such visualization 3006 may involve a 2D projection/dimensionality reduction to reduce the number of data dimensions and thereby provide visualization on a display screen or other 2D representation of for example annotations, protein signatures and clinical metadata. The refined annotations presented for visualization may include but are not limited to for example:
CD4 naive cells
CD4 memory cells
CD8 Tem (effector memory) cells
CD8 Tcm (Central memory T) cells
Monocytes
pDCs (plasmacytoid dendritic) cells,
MAIT (mucosal-associated invariant T) cells
Regulatory T (Treg) cells
Other.

Protein signatures may be displayed other otherwise represented with an indicator of high to low correlation, confidence, precision and/or reliability.

One example embodiment displays all the cells using a UMAP, which is a 2D visualization of similarly between cells. In an RNA based UMAP, if two dots are close together it means that they are similar in terms of RNA expression. In a CITE-seq-based UMAP, the meaning is they are similar based on surface protein expression. One embodiment uses those UMAPs for visualization, but projects more data on it such as annotation, signature score and/or expression of specific genes.

### Multiomic Characterization and Visualization of T Cells Using Heat Maps

A common method of visualizing gene expression data is to display it as a heatmap. A heat map is a graphical representation of data, where values are expressed as colors. A cluster heatmap is a heatmap where rows and columns of data of a data matrix have been ordered according to output from clustering. In one embodiment, cell type prediction processes are validated using RNA/protein pattern determining (e.g., heatmaps) per cell subset. For example, in multiomic characterization of T cells, a heatmap or other similar pattern determining technique may be used detailing more of the markers, specifically for the different T cell subsets. In one embodiment, the heatmap of T cell marker genes may be based on scRNAseq data. There may be a degree of overlap between the list of T cell marker genes examined by scRNAseq and the list of T cell marker proteins examined by CITE-seq, potentially providing redundant information. For example, in one embodiment, the heatmap of T cell marker genes is based on scRNAseq data, and there may be overlap between the list of T cell marker genes examined by scRNAseq and the list of T cell marker proteins examined by CITE-seq. The number of surface proteins that can be analyzed via CITE-seq started out being pretty small (on the order of 20-25, compared to the 35-40 genes shown in the heatmap figures), but currently can do many more. See, e.g., https://www.biolegend.com/en-us/blog/cite-seq-and-totalseq-reagents. Nevertheless, the number of surface proteins that CITE-seq can analyze is limited, so that scRNAseq may be provide information that is not available from CITE-seq.

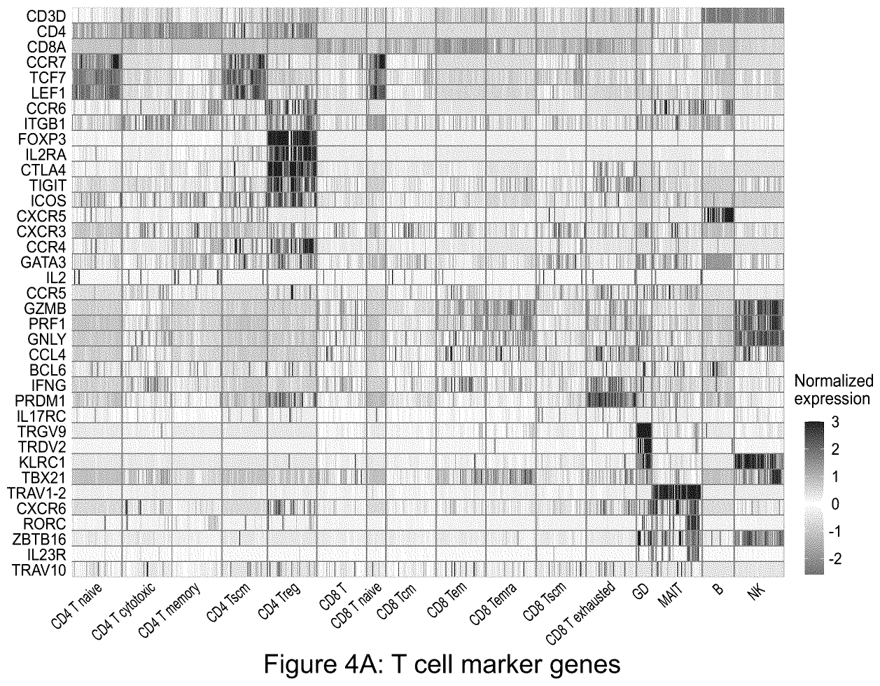

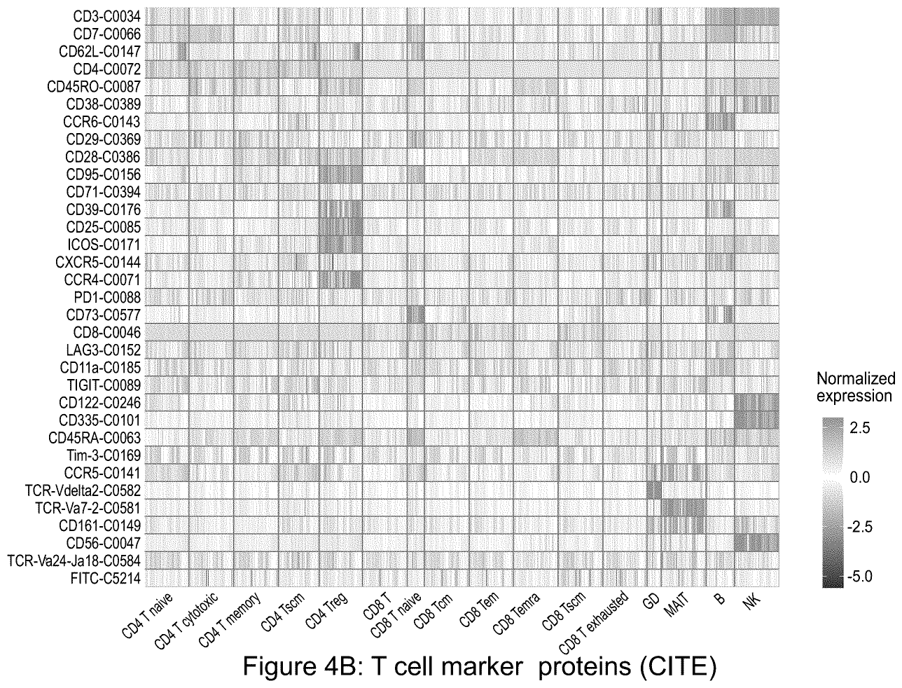

FIGS. 4A and 4B show heatmaps detailing more of the markers, specifically for the different T cell subsets. Thus the left-hand (row) legends indicate significant genes, and the columns delineate different T cell subsets or types. The black and white rendering of these heat maps will not clearly distinguish positive from negative normalized expression, so the reader is referred to the color versions of these drawings.

The heatmaps may also be combined with clustering methods which group genes and/or samples together based on the similarity of their gene expression pattern. This can be useful for identifying genes that are commonly regulated, or biological signatures associated with a particular condition (e.g., a disease or an environmental condition). See e.g., Grant, G. R., et al. (2007) Analysis and management of microarray gene expression data. Current Protocols in Molecular Biology Chapter 19: Unit 19.6.

In more detail, the exemplary heatmaps shown in FIGS. 4A and 4B detail more of the markers, specifically for the different T cell subsets. The heatmaps in this example show Primary Bone Marrow Mononuclear Cells, Normal, Human (BMMC). The heatmap of T cell marker genes in FIG. 4A may be based on scRNAseq data and the heatmap of T cell marker proteins in FIG. 4B may be based on CITE-seq. The heatmap in FIG. 4B thus shows the same T cell types in the columns as the FIG. 4A heatmap, but is in this case based on the CITE-seq technology that enables one to look at intracellular gene expression and extracellular protein marker expression simultaneously at a single-cell level of resolution.

In this particular example, the columns correspond to the following T cell subsets or types:
CD4+ T naive
CD4+ T cytotoxic
CD4+ T memory
CD4+ Tscm (Stem memory T cells)
CD4+ Treg
CD8+ T
CD8+ T naïve
CD8+ Tcm (Central memory T)
CD8+ Tem (T effector memory)
CD8+ Temra (T effector memory CD45RA)
CD8+ Tscm
CD8+ T exhausted (Exhausted T cells are characterized by progressive loss of effector functions (cytokine production and killing function), expression of multiple inhibitory receptors (such as PD1 and LAG3), dysregulated metabolism, poor memory recall response, and homeostatic proliferation.)
GD (gamma delta T cells) (the majority of T cells including nearly all that are listed above are T-alpha/beta)
Mucosal-associated invariant T cells (MAIT)
(the type of cells that produce antibodies)
NK (Natural Killer T) (Note: Natural Killer (NK) Cells are lymphocytes in the same family as T and B cells; TNK cells are distinct type of T cells characterized by combined expression of T cells and NK cell markers).

The expression patterns these two heat maps show are different but there is a certain amount of overlap between the list of T cell marker genes examined by scRNAseq and the list of T cell marker proteins examined by CITE-seq. For example, both heat maps highlight some of the CD4+ Th1 markers from the RNA, as well as few exhaustion markers which are high in the exhausted CD8+ T cells. Automatic analysis can in this way be used to detect common markers between genes and proteins. The annotation may thus be validated by, among other things, using RNA/protein heatmaps per cell subset.

Matching Clinical Signatures with Perturbation Signatures

Some in the past have combined CRISPR-based perturbations with single-cell RNA sequencing (scRNA-seq). See for example Burgess, “Combining CRISPR perturbations and RNA-seq”, Nature Reviews Genetics volume 18, page 67 (2017). The example technology herein extends such techniques to multiomics and cell type specific matching of clinical signatures. As discussed above, example embodiments herein combine clinically-derived information with multiomic sequencing and cell type prediction information and associated annotations. One embodiment performs cell type specific matching of clinical signatures with perturbation signatures.

In one embodiment, cell type-specific clinical signature from e.g., literature or in house cohorts, is validated and refined using public or other datasets. Signature is mapped against large scale CRISPR (clustered regularly interspaced short palindromic repeats) perturbations in a relevant cell type to identify potential drug targets. Receiver-Operating Characteristic (ROC) curve analysis may be used to determine sensitivity (true positive rate or TPR) vs. specificity (1-FPR, where FPR is false positive rate) of an automatic classifier.

In one embodiment, T cell clone tracing is coupled with clinical and phenotypic associations. As above, cell types are annotated in a multiomic approach, and clones of a specific cell type/cell type groups are grouped by their longitudinal trajectories (based on clinical data) over time. In example embodiments, some longitudinal trajectories are enriched for certain clinical covariates (response, treatment). In some embodiments, trajectories are also enriched in specific molecular phenotypes of interest (T cell exhaustion).

In one embodiment, T cell clonal dynamics associate with an immune response(s). Longitudinal clone tracing enables identification of a response signature. Longitudinal clustering analysis identifies a number of distinct clone trajectories, some of which are enriched in responders, some of which are enriched in non-responders. In one example, response associated trajectories are enriched in CD8 positive (CD8+) T effector memory cells expressing CD45RA (TEMRA) or other cells at certain time periods.

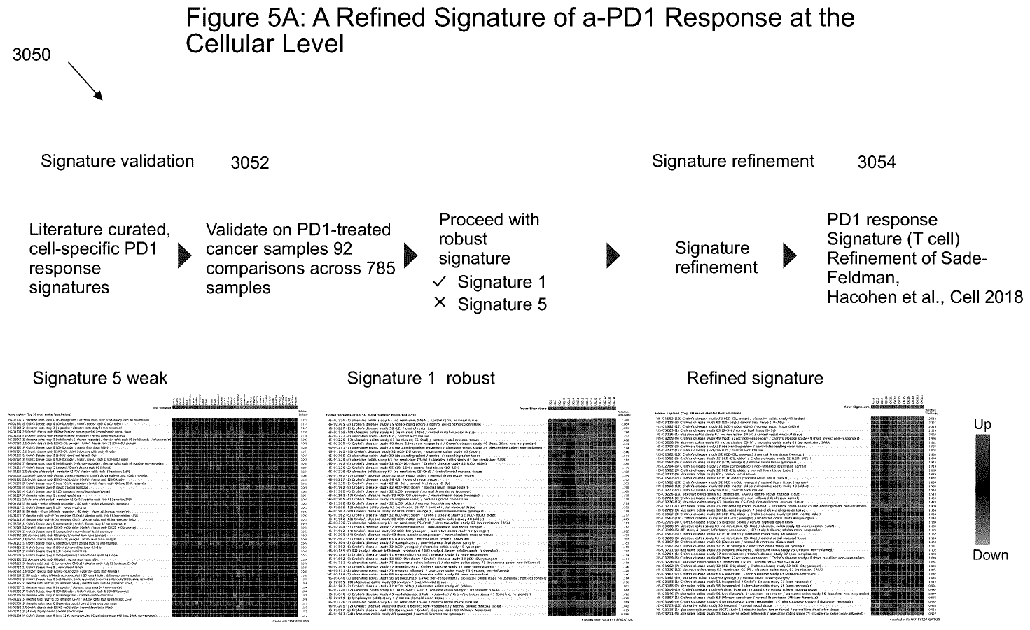

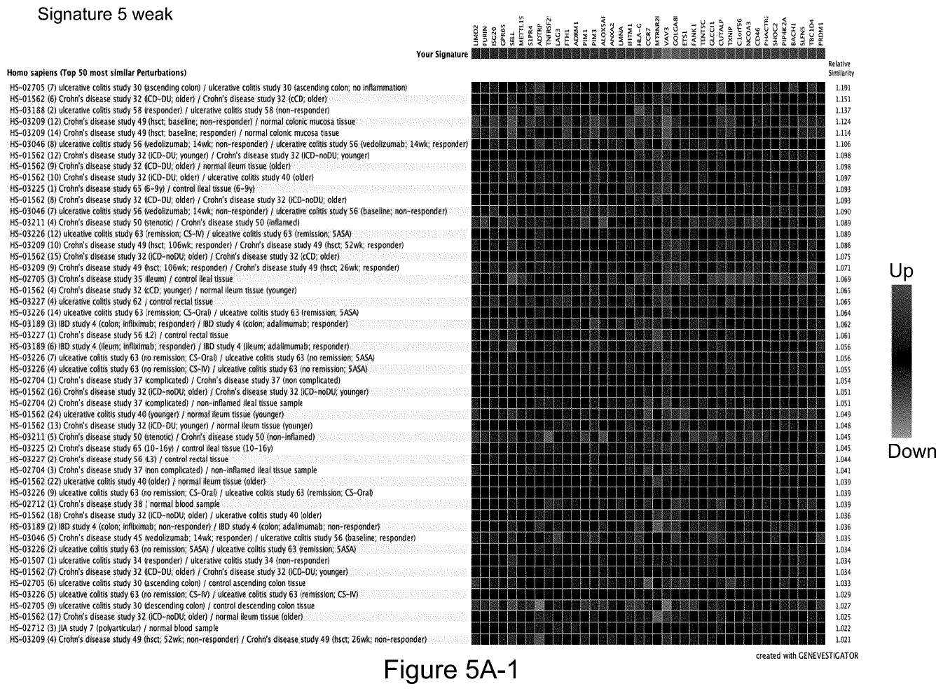

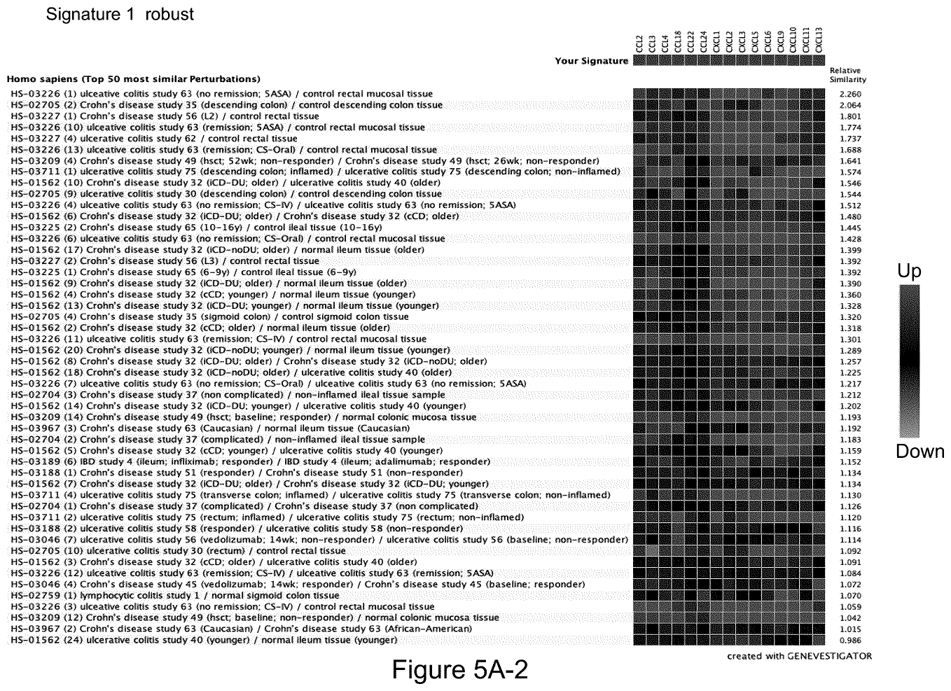

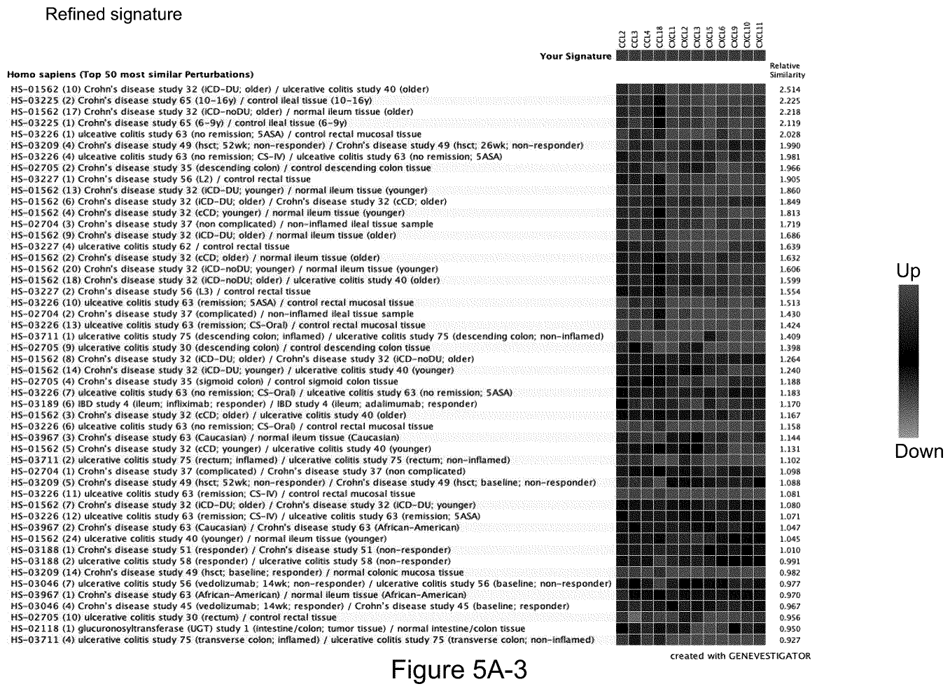

FIG. 5 shows a flowchart of exemplary cell type-specific matching of clinical signatures with perturbation signatures. Cell type-specific clinical signatures from literature or in house cohorts are validated and refined using public datasets (“signature validation” 3052) and the signature is mapped against large scale CRISPR perturbations in relevant cell types to identify potential drug targets (“signature refinement” 3054).

FIG. 5A, 5A-1, 5A-2, 5A-3 show a refined signature associated with a-PD1 response at the cellular level. See e.g., Riley, “PD1 signaling in primary T cells”, Immunol Rev. 2009 May; 229(1): 114-125, doi: 10.1111/j.1600-065X.2009.00767.x. Literature curated, cell-specific PD1 response signatures are validated on PD1-treated cancer samples; in this embodiment, the validation comprised 92 comparisons across 785 samples. Signature validation 3052 proceeds in this example with robust signature 1, thus discriminating weak signature 5. The PD1 response Signature (T cell) may be refined using techniques described in Sade-Feldman et al, “Defining T Cell States Associated with Response to Checkpoint Immunotherapy in Melanoma”, Cell 2018 Nov. 1; 175(4):998-1013.e20. doi: 10.1016/j.cell.2018.10.038 (study of immune cell transcriptomes from tumors demonstrates a strategy for identifying predictors, mechanisms, and targets for enhancing checkpoint immunotherapy).

In more detail, validation in the example shown was performed on PD1-treated cancer samples for a certain number (e.g., 92) comparisons across a certain number (e.g., 785) samples. FIG. 5A-1 shows a weak comparison for signature 5, which may result in discarding the signature as not being valid. Signature 1, on the other hand as shown in FIG. 5A-2, is found to provide a robust comparison with may result in Signature 1 being validated. The processing then proceeds with the robust signature (e.g., Signature 1) to perform PD1 response signature (T cell) refinement (see FIG. 5A-3) as described above.

### Platform Use Case—Clinical Covariate Enrichment

Generally, a covariate is complementary to the dependent, or response, variable. A variable is a covariate if it is statistically related to the dependent variable. Thus, any variable that is measurable and considered to have a statistical relationship with the dependent variable would qualify as a potential covariate. A covariate is thus a possible predictive or explanatory variable of the dependent variable. Covariates may also comprise variables that affect a response variable, but are not of interest in a particular study or analysis.

Clinical covariates have traditionally been used to predict disease. For example, clinical covariates such as age, gender, tumor grade, and smoking history have been extensively used in prediction of disease occurrence and progression. On the other hand, genomic biomarkers may provide an alternative, satisfactory way of disease prediction. Recent studies show that better prediction can be achieved by using both clinical and genomic biomarkers. However, due to different characteristics of clinical and genomic measurements, combining those covariates in disease prediction is very challenging. See e.g., Ma et al, “Combining Clinical and Genomic Covariates via Cov-TGDR,” Cancer Inform. 2007; 3: 371-378 (covariate-adjusted threshold gradient directed regularization (Cov-TGDR) used to combine different types of covariates in disease prediction).

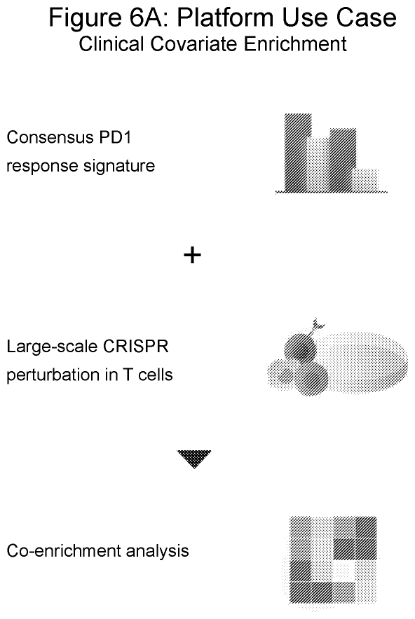

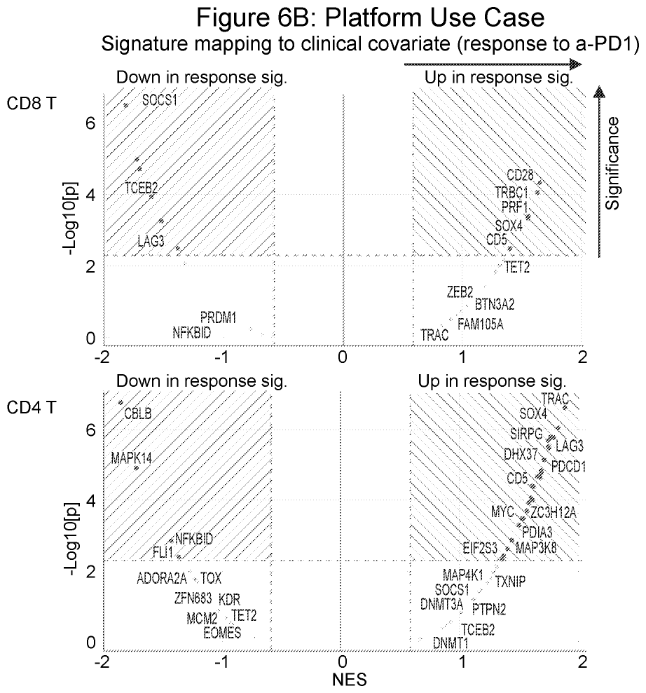

FIG. 6A shows an example platform use case including clinical covariate enrichment combined with the multiomics techniques described above. This particular example platform use case combines consensus PD1 response signature with large-scale CRISPR perturbation in T cells to provide co-enrichment analysis. In one embodiment, association of cell specific molecular phenotypes with a response of interest includes predicting cell types using a multiomic approach, and complex molecular phenotypes show association with clinical covariate (response).

Adaptive enrichment designs for clinical trials may for example include rules that use interim data to identify treatment-sensitive patient subgroups, select or compare treatments, or change entry criteria. A common setting is a trial to compare a new biologically targeted agent to standard therapy. See Thall, “Adaptive Enrichment Designs in Clinical Trials”, Annu. Rev. Stat. Appl. 2021. 8:393-411 https://doi.org/10.1146/annurev-statistics-040720-032818.

In the embodiment shown in FIG. 6A, a consensus PD1 response signature is developed as described above, and a large-scale CRISPR perturbation in T cells is performed. A co-enrichment analysis is then performed, mapping the PD1 response signature to clinical covariate (response to a-PD1).

FIG. 6B shows exemplary graphs of response signature mapping to clinical covariate (response to a-PD1). These graphs show CD8+ T and CD4+ T cell types with signatures that are down in response signaling to the left and signatures that are up in response signaling to the right. On each graph, significance increases as one proceeds upward in the vertical axis of the graph.

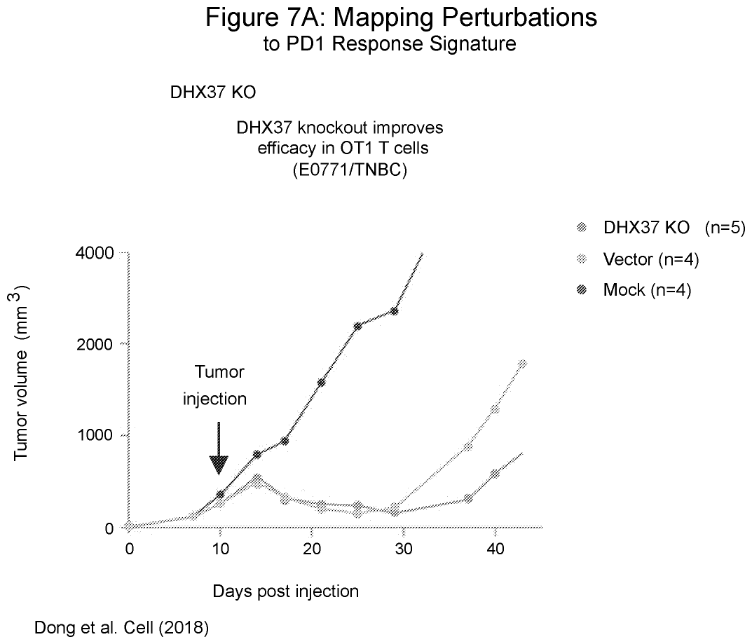

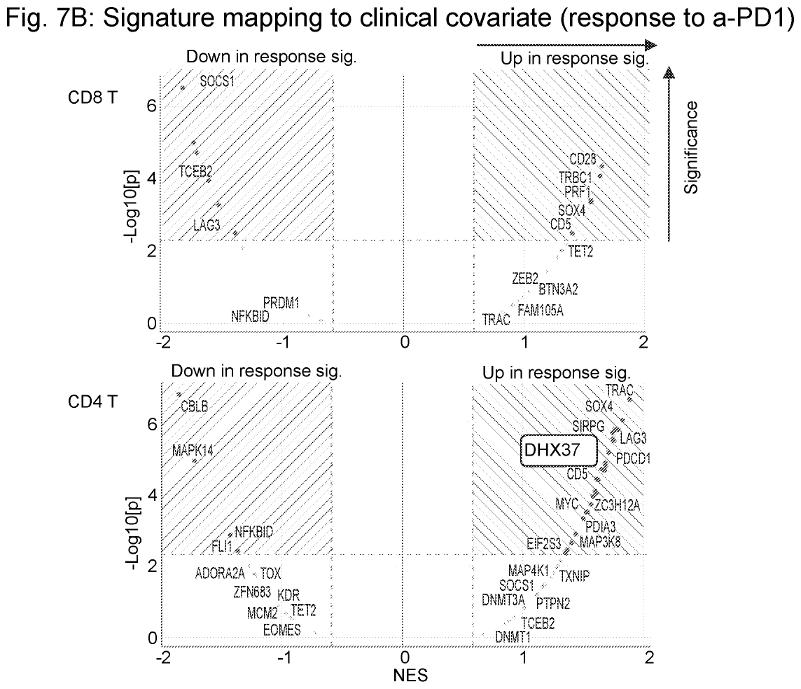

FIG. 7A shows one particular example focusing on a PD1 response signature for the gene DHX37. DHX37 knockout in CD8 T cells is known to enhance the efficacy of those cells following adoptive transfer. See Dong et al, “Systematic Immunotherapy Target Discovery Using Genome-Scale In Vivo CRISPR Screens in CD8 T Cells”, Volume 178, Issue 5, 22 Aug. 2019, Pages 1189-1204.e23. Note how DHX37 shows significance on the CD4 T graph of FIGS. 6B and 7B as a result of the covariate and co-enrichment analysis.

### Enriching Trajectories for Certain Covariates

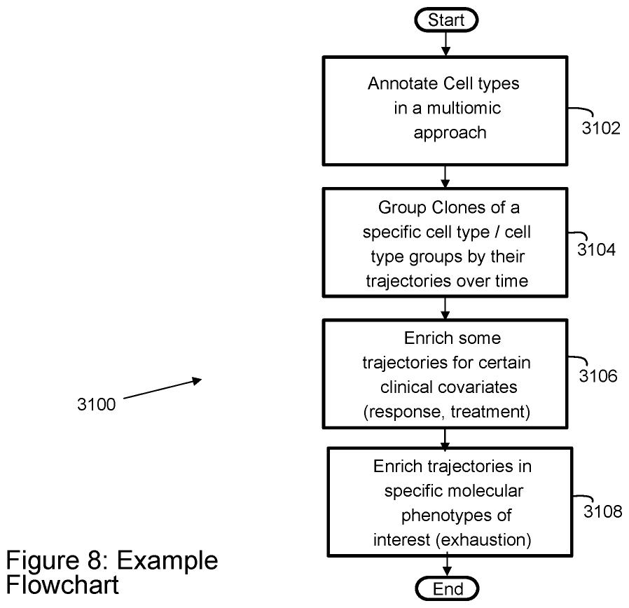

FIG. 8 is a further flowchart showing exemplary T cell clone tracing coupled with clinical and phenotypic associations. In this example process, cell types are predicted and annotated in a multiomic approach (3102) as described above. Clones of a specific cell type/cell type group are grouped by their trajectories over time (3104). Some trajectories are enriched for certain clinical covariates (response, treatment) (3106). Trajectories are also enriched in specific molecular phenotypes of interest (exhaustion) (3108).

FIG. 9 shows a series of exemplary graphs of T Cell Clonal Dynamics Associated with Response. In this example, longitudinal clone tracing enables identification of a response signature. Longitudinal clustering analysis identifies 8 distinct clone trajectories, 2 enriched in responders; and response associated trajectories are enriched in TEMRA CD8 T cells at Wk 3 (FU1). The results shown are an aggregate of all patients (13-14 patients), which is why abundance may exceed 100%. In the example shown, Trajectory 5 is associated with no response (NR) to therapy, and Trajectories 6 and 7 are associated with response (R) to therapy.

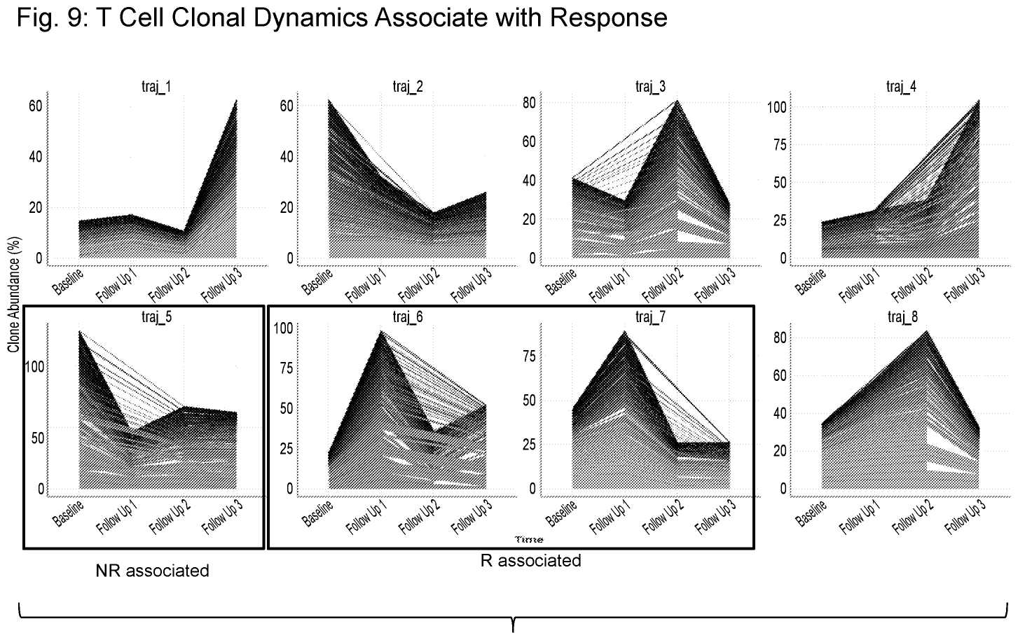

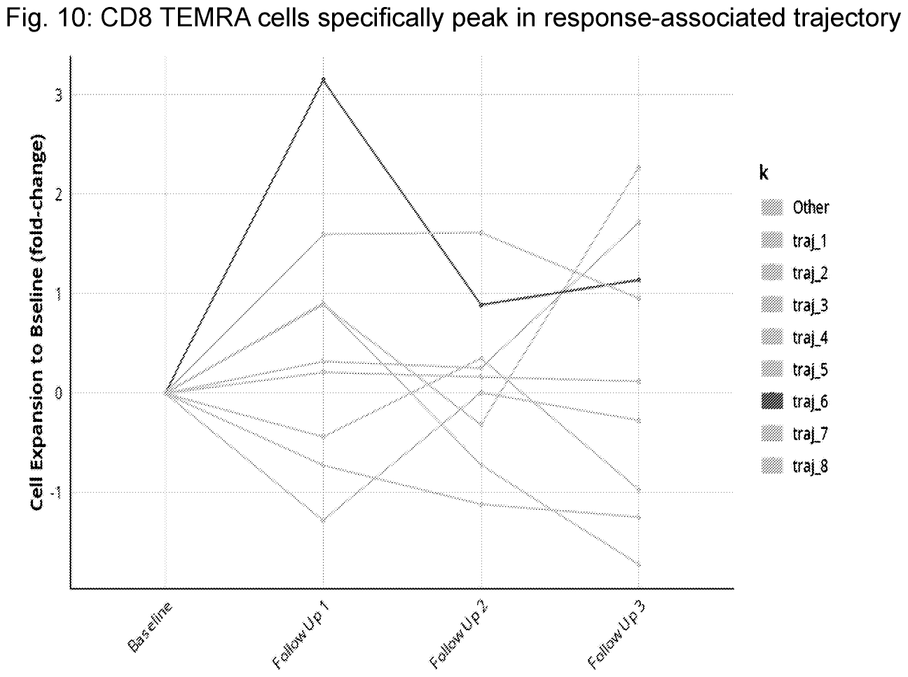

FIG. 10 shows that CD8 TEMRA cells specifically peak in response-associated trajectory 6 (darker trace).

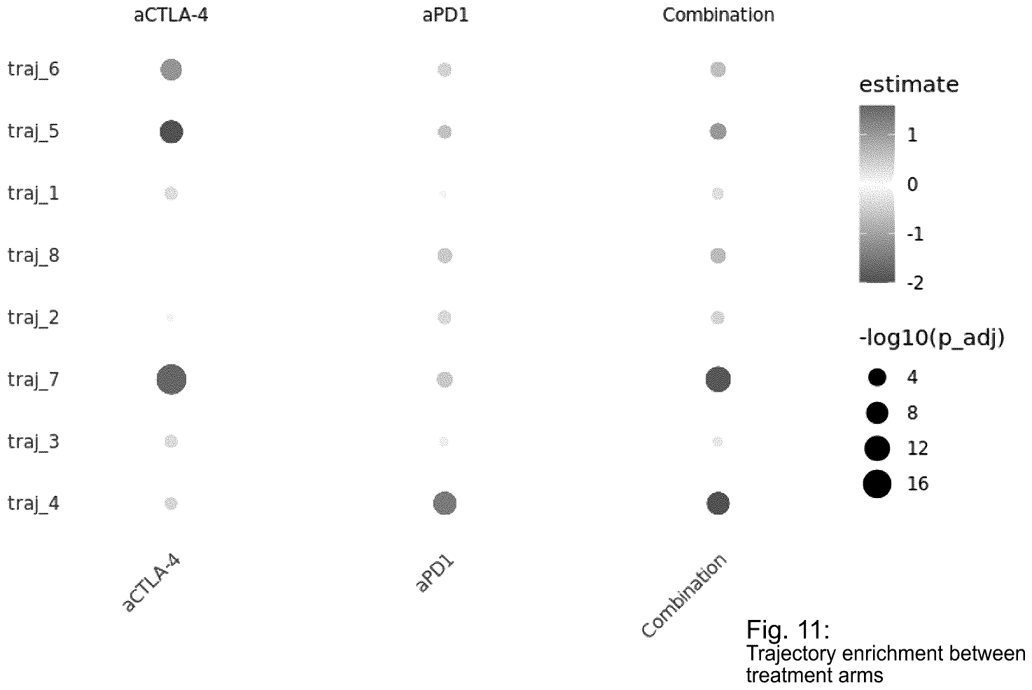

FIG. 11 shows exemplary trajectory enrichment between treatment arms anti-CTLA-4 (αCTLA-4) antibody and anti-PD1 (αPD1) antibody and the combination. In this drawing, the cross-hatched regions show positive estimates and the non-cross-hatched regions show negative estimates.

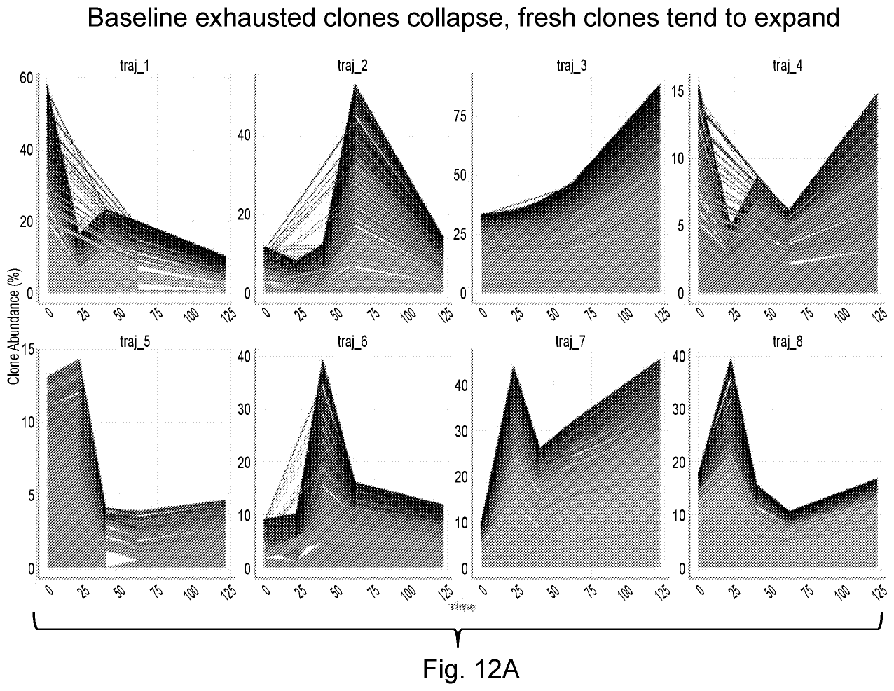

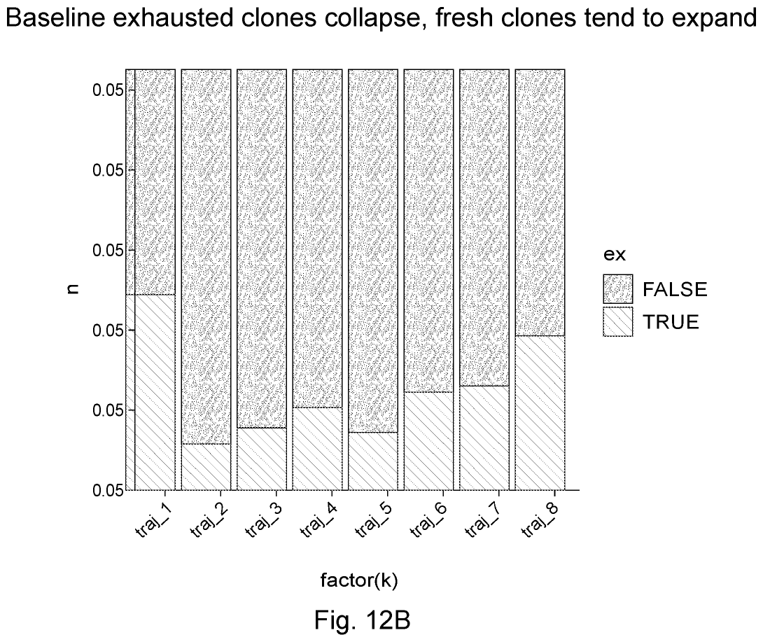

FIGS. 12A, 12B show that baseline exhausted clones collapse, whereas fresh clones tend to expand.

Associating Cell Specific Molecular Phenotypes with Response

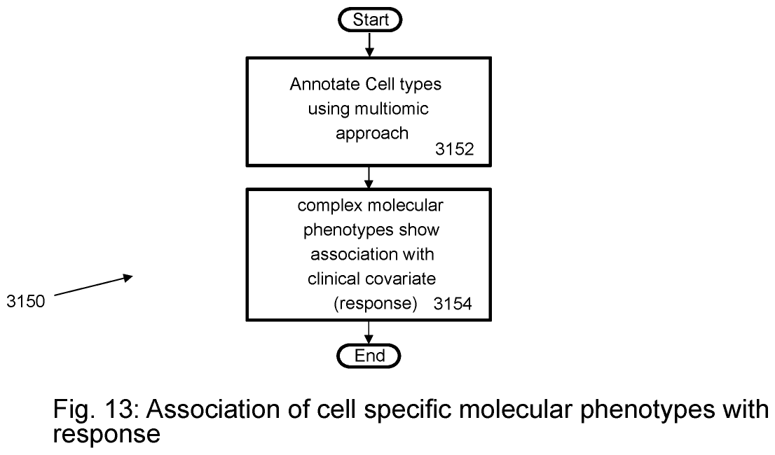

FIG. 13 shows an exemplary flowchart of a process of associating cell specific molecular phenotypes with response. In this example, cells are predicted and annotated using a multiomic approach (3152) as described above, and complex molecular phenotypes show association with clinical covariate (response) (3154).

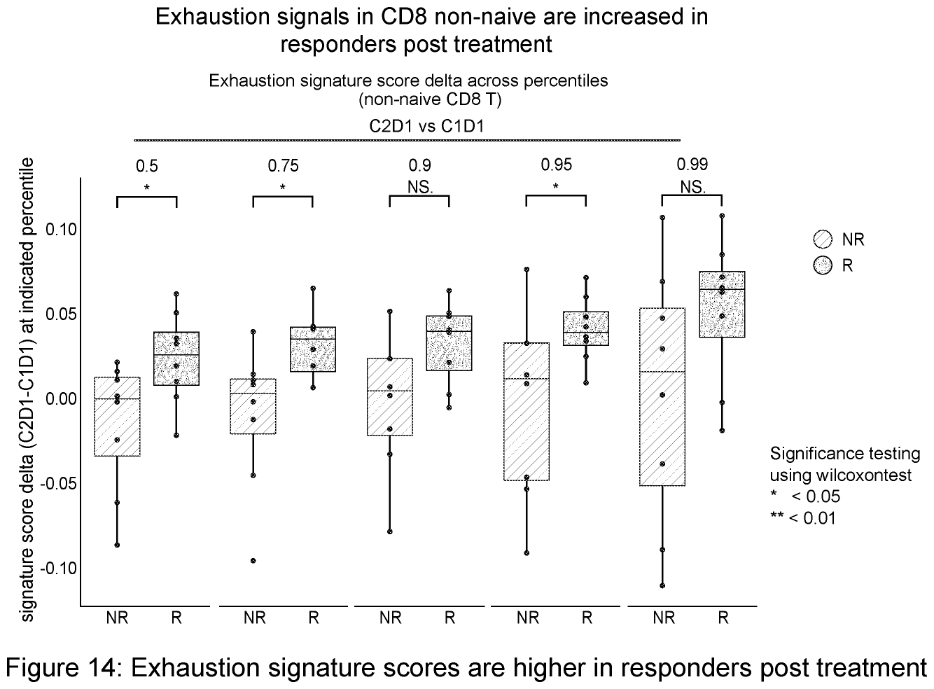

FIG. 14 shows an exhaustion signature score delta (δ) across percentiles (non-naïve CD8 T) and contrasts C2D1 vs. C1D1 for signature score δ at various indicated percentiles ranging from −0.10 to +0.10. Significance testing was performed using the Wilcoxon signed rank test (a non-parametric statistical hypothesis test used either to test the location of a set of samples or to compare the locations of two populations using a set of matched samples). FIG. 14 shows that based on such analysis, exhaustion signals in CD8+ non-naïve T cells are increased in responders post treatment (exhaustion signature scores are higher in responders post treatment).

### Exemplary System Architecture

### Architecture

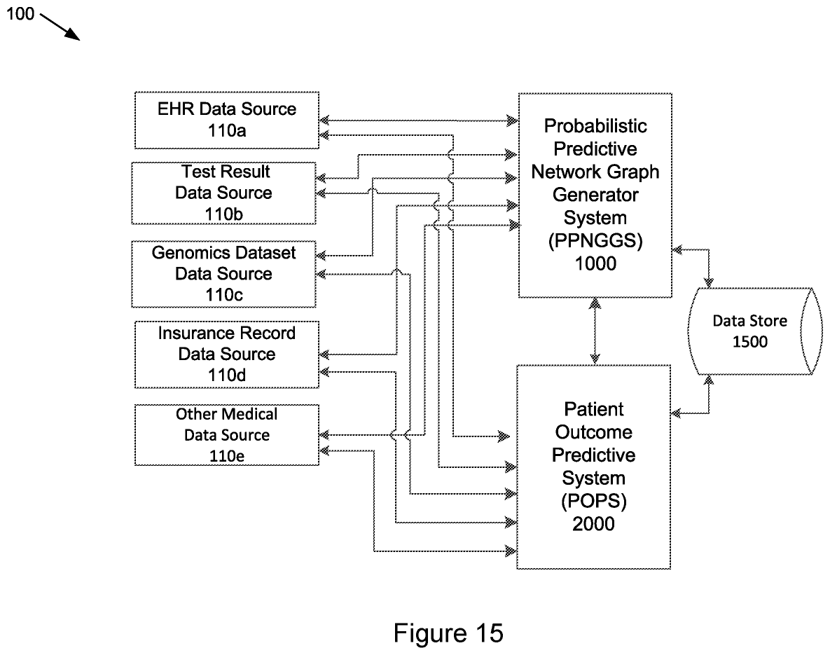

FIG. 15 is a schematic diagram of a predictor generation system (PGS) (100) structured to and capable of performing the various automated analyses and processing described above. PGS 100 comprises:
(1) a probabilistic prognosis graph generator system (PPNGGS) (1000) that collects longitudinal patient data and multiomic information, generates longitudinal trajectory information, and produces one or more graph representations,
(2) a storage system or data store (1500) in which these graph representations are stored, and
(3) a patient outcome predictive system (POPS) (2000) that produces medical treatment and prognosis predictions for a specific patient based upon at least the patient's medical and health information, one or more probabilistic prognosis graphs, and any previously saved prior predictions.

Generally speaking, the graphs that system 1000 produces are not only the graphs described above for visualization, but also data representation graphs that comprise sets of objects where some pairs of objects are connected by links. The interconnected objects are represented by points termed as vertices, and the links that connect the vertices are called edges. As discussed above, in exemplary embodiments the graphs are longitudinal in that they contain nodes measured at different times. FIGS. 20A, 20B and 20C show examples of such longitudinal graphs. See also, e.g., Liu, “Temporal Phenotyping from Longitudinal Electronic Health Records: A Graph Based Framework”, KDD '15: Proceedings of the 21th ACM SIGKDD International Conference on Knowledge Discovery and Data Mining, August 2015 Pages 705-714 https://doi.org/10.1145/2783258.2783352. As mentioned above, embodiments generate such graphs based on and to include a variety of different data types including but not limited to multiomics data and patient health records. The trajectories of such graphs may be predicted using machine learning including but not limited to neural networks or deep neural networks (“DNNs”) as shown by example in FIG. 17.

The probabilistic prognosis graph generator system (PPNGGS) (1000) and patient outcome predictive system (POPS) (2000) may each comprise one or more processors such as CPUs, GPUs, or other computation hardware and/or software, that execute instructions stored in non-transitory memory to perform tasks, operations and/or computations. Such processors may be distributed, operate in parallel or in series, execute concurrently or serially, and may be located anywhere including for example locally and/or in the “cloud” (e.g., remotely and communicating via one or more networks including for example the Internet). Such processors may be configured to provide machine learning components such as deep neural networks (DNNs) of various types that are trained in order to acquire weighted gain factors.

Each information source is operably connected to the PPNGGS 1000 using standard communications or networking technologies known to those skilled in the art. The storage system/data store (1500) may be any storage system able to store generated graph representations, such as relational or object-oriented databases, file stores, and similar storage systems, and may be part of the PPNGGS 1000 or may be a separate system from the PPNGGS. The PPNGGS 1000, data store 1500 and POPS 2000 may be located in the same installation or at different installations, and in some examples may be distributed. If required, the storage system/data store and the PPNGGS 1000 are operably connected using standard communications or networking technologies known to those skilled in the art.

In the example shown, the PPNGGS 1000 collects longitudinal patient information from a variety of information sources (e.g., information sources 110a, . . . , 100e) and processes the collected longitudinal patient information in order to produce an instance of a probabilistic prognosis graph representation, which is then stored in data store 1500. The probabilistic prognosis graph representation may include undirected or directed graph implementations. Generally speaking, directed graphs are a class of graphs that don't presume symmetry or reciprocity in the edges established between vertices whereas undirected graphs have an extra assumption regarding the reciprocity in the relationship between pairs of vertices connected by an edge (i.e., if an edge (a,b) exists between two vertices a and b, the edge (b,a) also exists). An undirected graph can be thus thought of as a directed graph by replacing each undirected edge by a pair of edges in opposite directions. Different data models may better fit directed or undirected graphs. Some graphs could comprise a hybrid of directed edges and undirected edges.

The information sources may in one embodiment be commercially available clinical, medical, and business information systems, including EHR, laboratory test results systems, insurance claim databases, and the like, as well as multiomics and immunology datasets (collectively as the multiomics data source 110c) including multiomics data and associated analysis and processing as described above.

The POPS 2000 is similarly connected to patient-specific information sources and collects current patient specific information, processes that collected information in order to determine the patient's current clinical and immune state, and then calculates and presents one or more probabilistically predicted outcomes of various treatment options based upon the patient's current disease and immune state and a probabilistic prognosis graph representation. The calculated predicted outcomes may be saved in a local data store and/or data store 1500 for subsequent comparison to updated predictions.

In one embodiment, the POPS 2000 is physically and/or electronically isolated from the PPNGGS 1000 so that confidential patient information available to the PPNGGS is not accessible by the POPS 2000. For example, firewalls, encryption, sandboxes, virtual machines and environments, and other conventional techniques can be used to prevent confidential patient information accessible to or present on the PPNGGS 1000 from leaving the PPNGGS to become available on the POPS 2000. In this way, POPS 2000 can be used by any user without compromising any legally protected confidential patient information used by, processed by or present on the PPNGGS 1000.

In more detail, the PPNGGS 1000 and the POPS 2000 may interoperate and interact with one another and may also share or communicate with one another via a data store 1500. Each of PPNGGS 1000 and POPS 2000 may receive data from various data sources such as an EHR data source 110a, a test result data source 110b, a multiomics dataset data source 110c (which may provide multiomics data as described above), an insurance record data source 110d, and another medical data source 110e.

Each of these system components are described in detail in sections below.

### Probabilistic Prognosis Graph Generator System (PPNGGS) (1000)

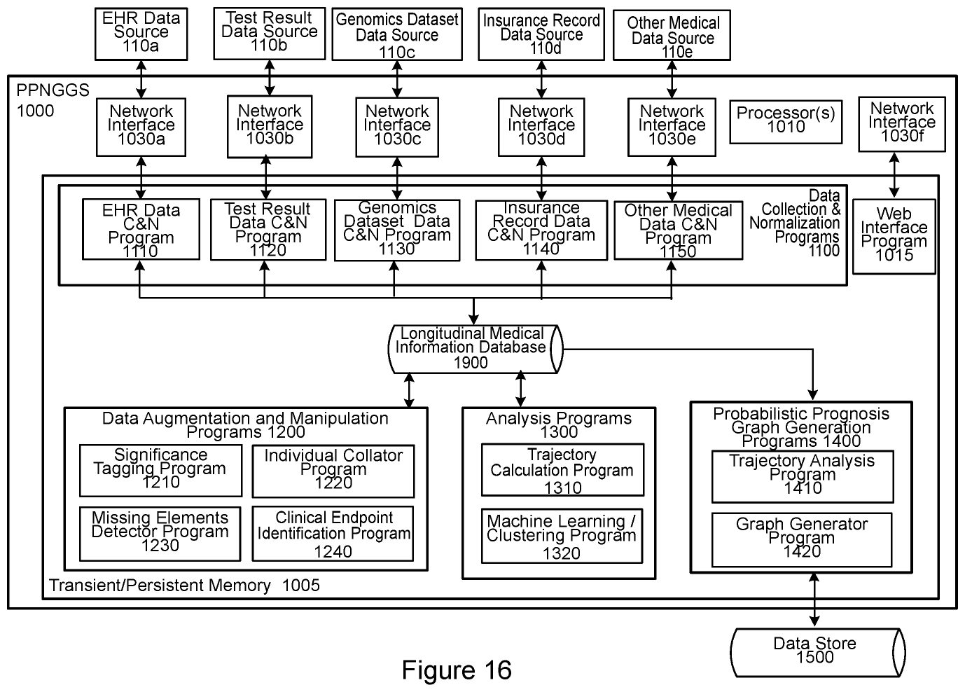

FIG. 16 is a schematic representation of an exemplary probabilistic prognosis graph generator system (PPNGGS) 1000 that collects longitudinal patient data, general genomic information from a variety of external data sources, and predicted cell types from multiomics as described above, and then processes the collected data in order to produce one or more graph representation(s).

The PPNGGS comprises at least one computer processor (1010) of standard manufacture, one or more non-transitory memories, of either volatile and/or non-volatile type (1005) for storing programs and data, and one or more network interfaces (interfaces 1030a, . . . , 1030f), shown here as separate interfaces for purposes of functional clarity, for use in operably connecting the PPNGGS programs or functions to external data sources or data stores, and for other connectivity-related purposes. The separate interfaces shown may be combined as desired without detracting from the operation of the PPNGGS 1000. While the processor 1010 may execute one or more application programs under the supervision of one or more operating systems of conventional design, the operating system program(s) are not shown for clarity.

The PPNGGS 1000 in one embodiment comprises one or more computer servers executing customized programs that control the processor 1010 in order to convert the processor of the computer server from a general purpose computer into a dedicated computer-controlled probabilistic prognosis graph generator system that automatically produces transferable probability prognosis graph representations that represent the set of longitudinal immune trajectories determined by the system and the probability of each branch in the graph being traversed. The programs are stored in or executed in volatile or non-volatile non-transitory memory (1005) of the server, and perform collection processing steps on data stored in or executed in volatile or non-volatile non-transitory memory, a data store of the system, and/or, in a particular embodiment, a longitudinal medical information database (1900).

The programs of the PPNGGS 1000 comprise a set of data collection and normalization programs (collectively 1100), a set of data augmentation and manipulation programs (collectively 1200), a set of analysis programs (collectively 1300), and a set of probabilistic prognosis graph generation programs (collectively 1400), each of which is operably connected to one or more of the data stores of the system (e.g., a database such as a longitudinal medical information database (1900)). Each of these components will be described in detail below.

The PPNGGS 1000 further optionally comprises a web interface program (1015) operably connected to a network interface (1030f). This interface may be used to configure or troubleshoot the PPNGGS 1000 and provides an optional user interface for applications operating on the PPNGGS.

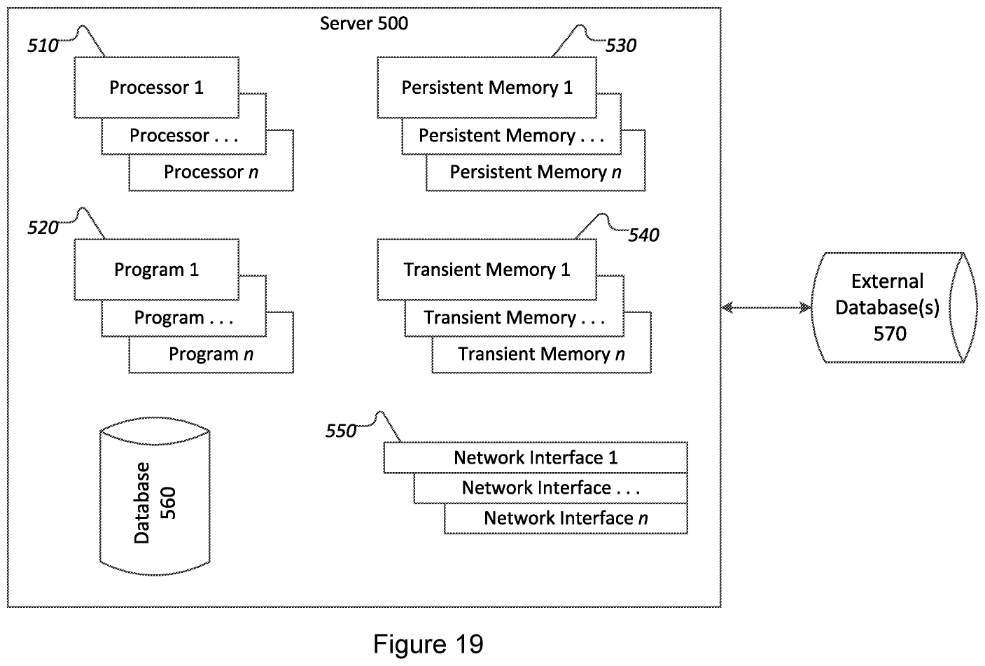

The longitudinal medical information database (1900) provides for the persistent storage of data collected and processed by the PPNGGS 1000. It is constructed using a data storage/database program as described in FIG. 19. One or more programs of the PPNGGS 1000 are operably connected to an external data storage system (data store 1500) for the storage of the graph representations produced by the system.

The PPNGGS 1000 in one embodiment undertakes the following general processing steps by executing instructions stored in non-transitory memory in order to collect and process longitudinal data and to produce a probabilistic prognosis graph definition:

1. Collects and preprocesses the collected data to generate processed collected data (e.g., data which is collected, de-duplicated, and associated with a person, a clinical/disease state, and/or an outcome. If necessary or desirable, further processing is performed to extract information embedded in the collected data (e.g., image analysis) during this step.

The collected data is optionally converted into formats that are usable by the other programs of the PPNGGS 1000 utilizing the further processing steps detailed herein, thereby creating a unified set of processed collected data.

The PPNGGS 1000 stores the processed collected data in a longitudinal medical information database tagged and associated in ways that automatically associate the processed collected data with existing data structures used by the patient outcome predictive system (2000). For example, new patient information is aggregated with existing data previously stored for a patient. In this way, the stored data is identified and formatted in a manner that allows efficient further processing of the stored processed collected data by the system.

2. Augments and/or enhances and/or enriches the processed collected data with immune state information as described above (e.g., genomic, transcriptomic, and proteomic data based on multiomics), clinical inferences (e.g., identifying conditions when toxicity occurs, adding missing clinical annotation tags, etc.), additional collected data, and identified clinical events and endpoints in order to produce augmented stored data. The augmentation processes are implemented by the programs of the data augmentation and manipulation group, in which the processed collected data is augmented by each program. In no particular order and without limitation, the stored processed collected data is:
A) cleaned and filtered to remove partial records and identify missing records by determining if one or more required data elements (such as dates for items of significance to be included in a future immune trajectory) are missing from the dataset and flagging those missing elements, and optionally obtaining additional collected data to complete these missing items which may potentially include imputation of missing sequences in addition to other clean-up step;
B) augmented by inserting tags and/or additional information associated with data elements of clinical or immunological significance, including imputing additional genomic references (sequences and/or gene identification) into the transcriptome identified by clinical tests in order to more fully complete the transcriptome;
C) cross-correlated with data from various different data sources to associate records belonging to specific individuals; and
D) augmented by identifying and tagging clinical and immunological endpoints. If -omic state augmentation is desired or required, that augmentation is performed as part of this step.
E) Stored as a data structure in a data store, creating augmented stored data in the longitudinal medical information database (1900),
for example, as described above in connection with FIGS. 3-14.

3. Determines clinical and immune trajectories from the augmented stored data by identifying records associated with a specific unique person, ordering these records in temporal order, adding trajectory associations to the ordered data so the determined clinical trajectory is repeatable, and tagging the trajectory relevant portions of the data records (such as outcomes/clinical endpoints) related to clinical and immune data. Incomplete trajectories are identified and tagged during these processes. If the same augmented stored data associated with a specific unique person could produce different trajectories, it may be desirable to add the trajectory associations to generate a specific trajectory again from the same data. These operations are performed by one or more of the analysis programs (1300) that are executed to compute the longitudinal immune trajectories and clinical trajectories based upon the augmented stored data and the determined trajectory information (in the form of clinical and immune trajectory data structures) is stored in the database, based for example on the flowcharts of FIGS. 3, 3A, 5, 5A, 6A, 8, & 13.

4. Processes the immune trajectory data for completed trajectories in order to determine clusters of immune states as described above for example using a cell differentiation trained Variational autoencoder (“VAE” (or other trained machine learning based system)) and defining specifications for each immune state in respect to observed dimensional traits. See, e.g., Davidsen et al., “Deep generative models for T cell receptor protein sequences”, eLife 2019; 8:e46935 DOI: 10.7554/eLife.46935. In some embodiments, the augmentation step may be performed in whole or in part by a trained machine learning program and may be combined in whole or in part with the VAE processing described herein. The system processes the augmented stored data using a machine learning program to determine cell types, homology, clinical states, and/or immune states in order to identify groupings, patterns and states in the augmented stored data. The definitions for the identified state groupings are then stored in the database associated with the augmented stored data. Using a VAE in this manner results in a reduced dimensional state definition for each of the clinical and immune state groupings, which reduces the amount of computing required to determine immune states. However, types of autoencoders or other machine learning structures and models other than VAE may be used.

5. Overlays previously determined trajectories with the VAE-identified immune/clinical state clusters/definitions to identify trajectories that pass through each identified immune state cluster and clinical state cluster. This step defines nodes and edges of a graph with respect to each of the defined trajectories.

6. Determines a probabilistic prognosis graph based upon the defined trajectories, such that each graph node is associated with a unique state cluster specification, and each edge is defined by an edge specification that describes a set of one or more trajectory paths from a first state to a second state along with the allowable variances to the trajectory paths. The lengths of the various trajectory paths and any intermediate states are added to the graph by this step (the path length corresponds to the number of edges in the path).

For example, the system first identifies the stored trajectory information and associates the stored trajectory information with the stored identified immune state definitions to identify which trajectories intersect specific immune states. These associations are then stored in the database on data store (1500). The stored trajectories are further analyzed in order to determine the common immune states and the portions of the trajectory associated with each immune state and the transition conditions that caused each trajectory to transition from a first immune state to a second immune state. These transition conditions may include specific treatments, the passage of time, and/or other events identified in the medical records associated with the immune trajectory(ies).

7. Determines the probability of and clinical/immunological event(s) associated with each immune state transition along an edge. Each trajectory in the set of trajectories is traversed in a certain order such as a reverse order, a forward order or some other order; and transition events/transition counts are accumulated for each state/transition along an edge. The transition events, their respective edges, and associated counts are then stored in the database associated with the immune states and immune trajectories. A graph representation is generated, with each node of the graph being a state as defined by a stored immune/clinical state definition, and each transition edge from a first state to a second state in the graph is represented by a description of the set of trajectory segments that make up that transition, along with enabling event definitions (e.g., treatment definitions/tags) and a count of the number of trajectory transitions encountered between these states in the graph for that event definition. In this way, a probability of transition from a first state to a subsequent state along a specific edge may be computed by calculating the ratio of the event count for the desired edge divided by the event count for all edges transitioning from the node. The graph(s) may be constructed in a variety of ways including but not limited to through use of machine learning. See, e.g., Hamilton, Graph Representation Learning (Synthesis Lectures on Artificial Intelligence and Machine Learning), Morgan and Claypool (2020); and Bollabos, Modern Graph Theory (Springer 1998), each incorporated herein by reference.

8. Creates a definition and/or specification of the probabilistic prognosis graph in a database by storing the graph representation to a data store (1500) for subsequent use by the POPS (2000). The definition/specification includes a definition for each node and edge of the graph, including the event transitions and the probabilities for each event transition to occur. Some variability of the node and edge definitions is expected and encoded within the graph definition. Note that the probability calculated in one embodiment is not the probability of the event, but the probability that the state will transition from the first state to the second state along the defined trajectory (edge) in response to a particular stimulus (e.g., an event associated with a specific treatment) or in the absence of any particular stimulus (i.e., when a treatment or other intervention is not administered).

### Data Sources of the PPNGGS (110)

The PPNGGS (1000) interacts with external data sources (110a-e) to collect relevant information from these sources for use in constructing the probabilistic prognosis graph representations. Each of these interactions is typically performed via a network interface using a distinct collection and normalization program as described for data collection and normalization programs (1100). Each type of collection and normalization program may be implemented as multiple instances, differently customized for the specific data source it is designed to interact with, or a single instance may be used without departing from the described system.

The EHR data source (110a) includes any patient clinical data stored in electronic form, such as by a doctor's office, clinic, hospital, urgent care, lab, or other health facility. This information includes one or more of patient demographic data, patient medical diagnosis and treatment records (including both current and historical records for procedures, drugs prescribed, vaccinations received, etc.), laboratory results, treatment outcomes, medical images (or parameters extracted from medical images, such as tumor size and location), and similar disease-associated data. EHR data may be distributed across a plurality of EHR data sources, and the data contained in one data source may overlap with the data contained in other data sources, as described below. The system uses the EHR data to determine the patient's personal and demographic data, clinical events, diagnosis, and treatment information related to various diseases and medical treatments that the patient is currently undergoing or has undergone in the past, and to construct clinical trajectories from this data. The clinical trajectories are used in conjunction with the immune trajectories to construct the graph representation outputs of the system.

The test results data source (110b) includes data related to types of medical tests, ranges of medical test results, and other more sophisticated assays including, for example, transcriptomic, genomic, and proteomic data (“-omic” data) that indicate various medical conditions. In particular, embodiments may use multiomic data as described above. Multiple sets of test results may be included in each test results data source. Test results data may be distributed across a plurality of test results data sources, collected directly from a testing machine, and/or may be included in EHR data sources described above. Additionally, the data obtained from one data source may overlap with the data obtained from other data sources, as described below. Such input data sources can include, but are not limited to, laboratory assays of blood, urine, and/or other bodily tissues and fluids, including lipid level, which may indicate for example presence or absence of specific protein and related metabolite levels, presence, absence, or circulating concentrations of various chemical compounds, chromosomal abnormalities, presence or absence of specific antibodies, genetic and tumor biomarkers (e.g. PD-L1, TMB, MSI, HLA), partial or complete transcriptome data, and multiomic data for immune cells found in tissues. Various other -omic data types may be related to the transcriptome data, or that they may be related to one of the other tests referenced in this list. In some cases, additional assays may provide more information about the patient's immune state.

The multiomic dataset data source (110c) includes data related to medical conditions that are caused or exacerbated by particular genetic traits or disorders, as well as information related to diseases associated with specific patient demographics. This data may be stored in one or more disparate datasets including for example genomic data, i.e., data that refers to the genome and DNA sequence of an individual patient. Genomic datasets may be obtained from a plurality of genomic dataset data sources, and the data contained in one system may overlap with the data contained in other data sources, as described below. Such genomic data can include, but is not limited to, cell type proportions (ratios) and classifications, and the proportions and classifications associated with specific disease and/or immune states, as well as databases that define transcriptome augmentation rules. However, as described above, the analyses performed by embodiments herein are not limited to genomics data sources, but instead use approaches in which the data sets are derived from multiple “omes”, such as the genome, surface proteome, transcriptome, epigenome, metabolome, and microbiome (i.e., a meta-genome and/or meta-transcriptome, depending upon how it is sequenced). Microbiome diversity is usually determined by sequencing conserved portions of the 16S rRNA, so it may be a very limited ‘transcriptome’ analysis. And embodiments herein focus on multilevel single-cell data, called single-cell multiomics by measuring tissue on a cell-by cell basis instead of or in addition to bulk assays. The single-cell assays are measured with multiple modalities—integrating many different -omics including genomics, single-cell RNA sequence data, surface proteome data, and other assays. Each of these assays provides a different perspective on the cell. By measuring more and more -omics over tens of thousands of different cells, it is possible to assemble a full profile of a patient's immune system.

The insurance records data source (110d) includes actuarial data acquired from insurance companies, including morbidity and mortality data for various medical conditions, as well as treatment and outcome data for various medical conditions encoded in machine readable form. Insurance records datasets may be distributed across a plurality of data sources, and the data contained in one system may overlap with the data contained in other data sources, as described above.

The other medical information data source (110e) comprises any sources of data that do not fall into any of the other data categories (e.g., 110a-d).

### Data Collection and Normalization Programs (1100) of the PPNGGS

The data collection and normalization programs (1100) of the PPNGGS (1000) are operably connected to one or more data sources (described above) and collect data from those data sources and pre-process/normalize the collected data for use by the system. The programs may use pre-computed filters or pre-computed transforms to completely process collected data at the time of collection instead of incurring the costs of saving the interim results to the longitudinal medical information database and then starting additional programs that read the saved data and perform the filtering, association, and/or data transforms as necessary.

Data collection and processing is carried out by one or more processors (1010) of the server using one or more data collection and normalization programs specific to the type of information being processed. For non-limiting purposes of illustration, the pre-processing and filtering steps are described as performed by the data collection and normalization programs (1100), although aspects of filtering, tagging, and associating may be performed by any program of the server.

The PPNGGS data collection and normalization programs apply pre-calculated filters to processed collected data to remove data that is extraneous, erroneous, distorted, or otherwise unreliable in order to improve accuracy of the remaining data. Generally, the functions of the data collection and normalization programs may be described as a collection of one or more of the following:

A) Filtering is performed to remove incomplete or inconsistent data from the collected data, for example, data where a patient was lost to follow up after a treatment or that has incomplete test results that would prevent the collected data from being used to generate an immune trajectory for that patient.

B) Association generation is performed to identify patient-specific records that belong to a known patient already present in the database so that new records (or records from disparate data sources) may be associated in order to form a more complete medical and immune history. For example, the collected data for a patient from a first EHR may be associated with data for that patient collected from a second EHR of a specialist such as an oncologist, and the lab test records associated with the patient are associated with the previously associated (e.g., combined) EHR data. Together the combined medical records provide a more complete clinical history of an individual patient's diagnosis and treatment.

C) Auto-tagging is performed to automatically determine data tags needed by the machine learning processes from the collected data. For example, collected data that indicates a diagnosis or treatment may be automatically tagged so that they are properly identified in the immune state and immune trajectory machine learning processes of the system.

D) Data extraction. In some instances, the collected data is not in a form where it is immediately usable the by the system. In these cases, data analysis tools appropriate to the collected data type are used to convert the collected data into generated data derived from the collected data. Simple cases include using natural language processing (“NLP”) and character recognition programs to interpret medical record data that includes handwriting and/or image recognition programs to extract tumor information from images, etc. Any generated data is subject to association and auto-tagging as described above.

E) Error correction and/or removal. In some instances, data is collected that is clearly erroneous. The erroneous data is either automatically corrected (if the correction is understood) or discarded. For example, if a set of related test results from a single blood draw is coded with differing dates, the dates of the test results may be corrected to represent the date of the blood draw. In other cases, if the data collected is inconsistent with previously collected information, the program reconciles the inconsistency between the data sources. For example, if one data source is identified as authoritative, the data from that source is retained, or if two lab results produce differing results (such as a first test resulting in an inconclusive diagnosis and a second test from a differing source resulting in a conclusive diagnosis), the conclusive diagnosis would be retained.

F) Redundancy resolution: When data is collected from a plurality of information sources, often redundant data is encountered. The data collection and normalization programs remove redundant data when it is encountered. Additionally, in some instances, multiple sets of results may produce a plurality of results with differing accuracy. In these cases, inaccurate (or reduced accuracy) data may have its accuracy improved or the less accurate data deleted.

Each data collection and normalization program (collectively 1100), as described individually below, collects patient data from various data sources and then processes the collected data in order to filter, normalize, associate, and/or auto-tag as described above to produce processed collected data that is then stored to a persistent memory and/or longitudinal medical information database (1900). One or a plurality of instances of the program may be used. Each instance of the program may communicate with one or more data sources in order to receive and process data.

The EHR data collection and normalization program (1110) collects and processes patient data from various electronic data sources.

The test result data collection and normalization program (1120) collects and processes patient data from various data sources comprising laboratory and genomic test results.

The multiomics dataset data collection and normalization program (1130) collects and processes multiomics data from various data sources, including public or private databases, summarized outcomes from research, and similar sources, and assays of patients. In some implementations, the system obtains configuration weights and parameters from the multiomics datasets for use by the neural network processes of the system.

The insurance record data collection and normalization program (1140) collects and processes insurance actuarial and claim data from insurance record databases.

In addition to the exemplary types of data collected from exemplary data sources discussed herein, the PPNGGS (1000) can collect and process data from any data source, referred herein as other medical information data source (110e), that is configured to provide data.

### Data Augmentation and Manipulation Programs (1200)

The system further comprises a set of data augmentation and manipulation programs (collectively 1200) as described above that make determinations about the cleaned and normalized data stored in the longitudinal medical database, and then augment that data as described below to create augmented longitudinal data in order to make it ready for use by the analysis programs (collectively 1300) and the graph generation programs (collectively 1400), in order to create a probabilistic prognosis graph (PPNG)

The data augmentation and manipulation programs comprise programs that augment collected data stored in the database by adding missing information, inferring information about the data, associating related information not previously associated, and tagging relevant portions of the data with identified significance.

These programs include a significance tagging program (1210) that makes determinations about and writes tags associated with one or more data elements in the longitudinal medical information database. These tags are associated with and identify aspects of the collected data that are considered important or significant for further processing.

A second program, an individual collator program (1220), associates information related to specific individuals from differing data sources (and/or differing collection operations) so that all information for a specific individual is associated within the database. These associations are written to the database. An exemplary collator program matches patients by medical ID number across a plurality of information data sources and databases and ensuring that all collected data for that patient is associated as part of the record for that patient.

A third program, a missing elements detector program (1230), scans the stored data and identifies missing data elements. These missing data elements are either automatically or manually collected and processed, or the missing data element reference is tagged as being incomplete. The missing elements detector program optionally identifies possible data sources where the necessary data may be found. For example, test results from differing providers may be stored in different results storage systems and/or differing EHRs, and the missing elements detector program identifies the missing data and its likely data source from other data in the record, e.g., insurance records, or lab referrals. The generated tags are written to the database or data store associated with one or more incomplete data elements.

A non-limiting example of a missing element is a clinical record that indicates a test was ordered, but for which no test results can be found.

A fourth program, a clinical endpoint identification program (1240), identifies and tags clinical starting points and clinical endpoints and significant clinical events in the stored database based upon its stored configuration information. The generated tags are written to the database associated with one or more clinical events. An example of the program identifying significant clinical events in the stored database is the identification of the starting point of a clinical trajectory comprising one or more diagnosis and treatment events and a clinical endpoint event when the collected medical data indicates the patient has returned to normal. In a more complex example, a clinical starting point of a trajectory may include an initial diagnosis for a cancer, significant clinical events may include specific genomic testing, biopsy, and the associated results indicating the type of cancer cells and related immune response of the patient, a chemotherapy treatment, and a final clinical endpoint when the patient no longer exhibits the cancer cells and related immune response.

Collectively, these programs annotate the collected records to make the subsequent analysis processes more efficient and store the resulting data to one or more data stores.

### Analysis Programs (1300)

The analysis programs (collectively 1300) comprise a trajectory calculation program (1310) that associates disparate data elements within the database with one or more individual disease trajectories, and machine learning/clustering programs (1320) that utilize machine learning techniques to distinguish and identify the immune state of a patient.

The trajectory calculation program (1310) processes the stored, augmented data by identifying data associated with a unique person, placing the data in temporal order, adding trajectory associations to the ordered data, and tagging the trajectory relevant portions of the data (such as outcome/endpoints) related to clinical and immune data, and/or a mixture of clinical and immune state data. This process is performed on clinical and/or immune data in order to create the corresponding clinical and immune longitudinal trajectory representations. The trajectory information and associations are stored in the database associated with the original stored data, and trajectories that have missing or incomplete portions are tagged for exclusion from further processing. The longitudinal trajectories are discussed in greater detail below.

The machine learning/clustering program (1320) processes the stored trajectory data for completed trajectories using various deep neural network techniques. Machine learning/clustering programs may include one or more deep neural network and related computing architectures, including one or more of the following: convolutional neural networks (CNNs), recurrent neural networks (RNNs), reinforcement learning (RL), inverse reinforcement learning (IRL), deep reinforcement learning (DRL), generative adversarial networks (GAN), and GAN-like variants, including adversarial auto-encoders which include the variational auto-encoders (VAE) described above.

In a specific embodiment, the machine learning/clustering programs include a trained variational auto-encoder (VAE) neural network to distinguish groupings of the differing immune cell characteristics that make up the immune states, and to distinguish the differing immune and clinical states and relationships between them, and to characterize each state's reduced dimensionality. The trained VAE takes as input all of the stored data associated with each clinical and immune trajectory, including the augmented data, tags (including clinical and imputed tags), and relationships identified by prior processes, including the augmented transcriptome from each clinical test that produces one, and identifies the clustering of immune (and clinical) states. Each parameter of these state clusters is characterized in order to provide a “fuzzy” state definition for each of the immune states, and to identify the immune states from different trajectories that are related by certain immune state parameters. “Fuzzy” state definitions are used because of the individual variance in treatments and response of each patient. In this way, the immune states associated with a plurality of trajectories are identified and clustered and the parameters of those clusters are defined. A trained VAE provides unique efficiencies in identifying these clusters and results in cluster specifications of reduced dimensionality. Other machine learning systems such as GANs and GAN-like variants may be used in addition to, or in place of a VAE. A sample VAE representation, including its inputs and outputs, is illustrated in FIG. 17.

The system thus processes the augmented stored data using a trained machine learning program to determine similar cell types, clinical states, and immune states in order to identify similar cell type, clinical state, and immune state groupings within the augmented stored data. The definitions and characterization information for the identified state groupings are then stored in the database associated with the augmented stored data.

### Probabilistic Prognosis Graph Generation Programs (1400)

The probabilistic prognosis graph generation programs (collectively 1400) comprise programs that generate a directed probabilistic prognosis graph from the collected and augmented data.

The trajectory analysis program (1410) performs analysis on the stored trajectories and VAE-identified state clusters/definitions and identifies the trajectories that include specific immune state and clinical state clusters. This step then identifies the nodes and edges of the resulting graph (either longitudinal immunological or clinical graphs, which may be either directional or non-directional) with respect to each of the defined trajectories. For example, the system first identifies and associates the stored trajectory information with stored identified immune state definitions to determine which trajectories intersect specific immune states. These associations are then stored in the database. The stored trajectories are further analyzed in order to determine the common immune states and the portions of the trajectory associated with each immune state (and the transition conditions such as the administration of a specific drug that caused each immune state/trajectory to transition from a first immune state to a second immune state). These transition conditions may include specific treatments, the passage of time, or other clinical events identified in the clinical data associated with the immune trajectory(ies). The trajectory transition conditions are identified and tagged for later use by the graph generator program.

Similarly, each segment of the trajectory linking two states becomes part of a graph edge definition between the graph nodes that contain the linked states. The key features of each trajectory segment (e.g., elapsed time, variations in immune state) are identified and the trajectory segment annotated with this information. The trajectory state, segment annotations, key feature data, transition conditions, and tags are stored in the database for subsequent use.

The graph generator program (1420) generates various graphs and graph representations (e.g., a probabilistic prognosis graph, a longitudinal immunological directed graph, and a longitudinal clinical directed graph) from stored trajectory information. A generalized generation process is described below, the difference is the type of input data provided to the program (e.g., immune trajectory, clinical trajectory, or both). The graph generator program selects and reads all trajectories to be included in the graph generation process, then identifies the state cluster definitions associated with each trajectory. Selection of trajectories for inclusion in a graph may be based upon pre-defined selection criteria, such as the trajectory having one or more clinical event tags, a specific immune state, specific clinical state, and specific clinical data associated with it. The set of identified state cluster definitions are stored as node definitions of the graph. For each selected trajectory, the program traverses the trajectory from its end point to its starting point (e.g., in reverse order), visiting each traversed state cluster/node definition that is part of the trajectory. For each trajectory segment, an edge is defined in the graph representing that segment transitioning from a first node to a second node, and the edge is characterized with its attributes (minimum or maximum length (e.g., representing time), incorporated interim immune states, etc.), a set of one or more transition-causing events, and a use count set to 1. If the edge is already defined in the graph, a use count may be incremented or otherwise modified to change or augment the edge characteristics.

Once all selected trajectories are traversed, similar edge definitions are combined, and the respective use counts added together to produce a combined use count for each edge/trajectory segment. A specification for the edge is then generated that identifies the set of parameters that represent a transition from a first node/state to a second node/state along a particular trajectory segment. The node-associated state definitions, transition events, their respective edge definitions, and associated counts are then stored in a database associated with one or more of the clinical state, clinical event, associated immune state and immune trajectories.

A graph representation is then generated with each node of the graph being associated with a state as defined by a stored state definition, and each transition edge from a first state to a second state in the graph represented by a description of the set of trajectory segments that describe that transition, along with enabling event definitions (e.g., treatment definitions/tags) and a count of the number of trajectory transitions encountered between these states in the graph for that event definition. In this way, a probability of transition from a first state to a subsequent state along a specific edge may be computed by calculating the ratio of the event count for the desired edge divided by the event count(s) for all edges transitioning from the node.

The definition and/or specification of the probabilistic prognosis graph representation is then stored in a data store (1500) for subsequent use by the POPS (2000). The definition/specification includes a definition for each node and edge of the graph, including the event transitions and probabilities for each event transition to occur. The probability calculated is the probability that the state will transition from the first state to the second state along the defined trajectory (edge) in response to a particular stimulus (e.g., an event associated with a specific treatment). In some cases, the defined event may include a “no action” event that may be used to represent a decision to undertake no treatment or procedure.

### Patient Outcome Predictive System (POPS) (2000)

The Patient Outcome Predictive System (POPS) (2000) collects an individual's clinical and immune data and generates clinical and immune trajectories in the same or similar way that the PPNGGS (1000) does. The individual's clinical and immune data and trajectories may be collected and stored separately if a need for data and processing separation is present. Alternatively, the collection and processing of an individual's clinical and immune data and trajectory information proceeds in the same manner as described above for the PPNGGS (1000). For brevity, those descriptions are not repeated here.

The POPS (2000) provides a set of prognosis generation programs (2400) which utilize the previously stored graph representations to generate a probabilistic prognosis for an individual patient.

Once an individual trajectory representation is established in the database, the prognosis estimator program (2410) matches the calculated immune trajectory representation against the previously stored graph representation in order to determine where in the graph the individual's immune trajectory places them. The current state information is read from the graph representation and presented to the user using a user interface such as the web interface. The program then traverses the prognosis graph in a forward direction (e.g., toward the endpoints), totaling the prognosis probabilities at each node, to produce a list of prognoses and the associated probabilities. The resulting list of prognoses, the transitioning events (e.g., the administration of a drug or procedure), and the probability of a specific outcome are extracted from the graph representation, optionally filtered to remove certain data, and the remaining results used to calculate each path's probability based in part upon the individual's condition. These probabilities are presented as a set of treatment actions and outcomes, accompanied by the calculated path probability (i.e., the chance of a particular outcome occurring) of each particular outcome and the associated patient prognosis based upon the patient's current immune and clinical trajectory information.

Filtering of the prognosis table is a rule-based process, in which the prognosis table rows are matched against one or more rules stored in the system, and rows are included or excluded from the final presentation based upon matching one or more of the criteria defined in a rule. Exemplary exclusion rules include those that identify low risk/low probability trajectories, while exemplary inclusion rules include those that identify low probability/high risk trajectories. Broader exclusion/inclusion rules also may be defined, such as rules that exclude trajectories with a probability of less than a specified threshold, or to exclude trajectories that include a contra-indicated treatment and/or diagnosis. Similarly, rules may be configured that filter, at least in part, based upon the diagnosis and/or treatment(s) and probability of the trajectory, thus permitting the system to filter the prognosis for different diseases at differing user-specified threshold levels.

The filtering process results is a probability weighted prognosis listing with diagnosis and treatment events defined for each graph pathway, where the patient's current immune state and clinical information is matched to graph nodes, and the diagnosis/treatments associated with the prognosis are identified from the graph node definitions. The resulting prognosis is stored in a database or data store for subsequent reference.

When a graph representation or the individual trajectory information is updated, the system runs the Prognosis Updater Program (2420) that retrieves a previously generated prognosis, calculates a new prognosis as described above, and then compares the newly generated prognosis against the previously stored prognosis. The results of the comparison are presented to the user on a user interface.

In some implementations, the PPNGGS (1000) and POPS (2000) are combined for efficiency into a single server.

An exemplary computer server (500) is illustrated in FIG. 19. Each exemplary server comprises one or more processors or data processing components (510), operably connected to memories of both persistent (530) and/or transient (540) nature that are used to store information being processed by the system and to additionally store various program instructions (collectively referred to herein as “programs”) (520) that are loaded and executed by the processors in order to perform the process operations described herein. Each of the processors 510 is further operably connected to networking and communications interfaces (550) appropriate to the deployed configuration. Stored within persistent memories of the system may be one or more data stores used for the storage of information collected and/or calculated by the servers and read, processed, and written by the processors under control of the program(s). Data store (560) is an internal instance of at least a portion of the longitudinal medical information database (1900). A server may also be operably connected to an external data store (570) via one or more network or other interfaces. The external data store may be an instance of the longitudinal medical information database that is provided on another server or may be a network connected database from a commercial or other external source.

Persistent memories may include disk, PROM, EEPROM, flash storage, and related technologies characterized by their ability to retain their contents between on/off power cycling of the computer system. Some persistent memories may take form of a file system for the server, and may be used to store control and operating programs and information that define the manner in which the server operates, including scheduling of background and foreground processes, as well as periodically performed processes. Persistent memories in the form of network attached storage (storage that is accessible over a network interface) may also be used without departing from the scope of the disclosure. Volatile non-transitory memories may include RAM and related technologies characterized by the fact that the contents of the storage are not retained between on/off power cycling of the computer system.

One or more databases are stored within at least one persistent memory of the system and are logical parts of longitudinal medical information database (1900). These databases may include local file storage, where the file system comprises the data storage and indexing scheme, a relational database, such as those produced commercially by the Oracle Corporation or MySQL, an object database, an object relational database, a NOSQL database such as commercially provided MangoDB, or other database structures such as indexed record structures. The databases may be stored solely within a single persistent memory, or may be stored across one or more persistent memories, or may even be stored in persistent memories on different computers.

The server may also include message notification and alerting programs, which facilitate inter-process and inter-server messaging and notification systems, such as operating system provided inter-process communication facilities (IPCs) and third-party messaging middleware subsystems such as MQ from IBM. The server may also include utility program scheduling programs, such as “cron” on Unix systems or scheduled tasks on Windows systems, that are used to run specific programs on a periodic or scheduled basis.

The network interfaces (550) are operated under control of the processor and the processing instructions contained within the control and operating programs mentioned above. These interfaces provide a connection to wired and wireless networking products that operably connect the servers, data sources, and network services described herein.

The server supports one or more programs for providing server management information utilizing a web services interface or other dedicated management information reporting systems such as SNMP for purposes of providing management information useful to report on the operation of the server. For purposes of clarity, each network interface (550) is illustrated as a separate interface but may be implemented as one or more interfaces if desired.

### Processes of the Technology

### Cluster Diagram and Trajectory Production Using Machine Learning Techniques

### Probabilistic Prognosis Graphs

As previously described, a PPNGGS generates one or more probabilistic prognosis graphs based on longitudinal trajectories generated using individual trajectory information from multiple patients. Referring to FIG. 20A, two exemplary non-limiting probabilistic prognosis graphs (PPG1, PPG2) are illustrated. Both probabilistic prognosis graphs share a common starting point within a cluster of nodes (collectively called a starting point cluster (SPC)). The probability prognosis graph (PPG1) further includes the following nodes: state cluster (1-1) joined to starting point cluster (SPC) by edge (1-1), state cluster (1-2) joined to state cluster (1-1) by edge (1-2), and an endpoint cluster (EPC-A) joined to state cluster (1-2) by edge (1-3). Each of the nodes (SPC, 1-1, 1-2, 1-2, EPC-A) is characterized by a state cluster definition which defines a boundary that encompasses a set of states that comprise the cluster of states that are included in the node. The states of the cluster of states included in each node include one or more of immune states and clinical states. Each edge (1-1, 1-2, 1-3) is represented by a set of trajectory segments that make up a transition between states (e.g., edge 1-2 includes trajectory segments vector 1[1, 2] and vector 2[2,3]), enabling event descriptions, probabilities, and a count of state transitions between states for an n event definition. Each edge can include any non-negative integer number of trajectory segments 0-N where N in exemplary embodiments may be many more than two trajectory segments (not shown for clarity) the extent of which are illustrated by dashed lines representing probability prognosis graph (PPG1) variance (V1). Probability prognosis graph (PPG2) includes a starting point cluster (SPC), node state clusters (2-1, 2-2, and 2-3), and second endpoint cluster (EPCB) joined by edges (2-1, 2-2, 2-3, and 2-4) with the extent of trajectory segments corresponding to the edges represented by variance (V2).

The starting point cluster (SPC), multiple endpoint clusters (EPCA, EPCB), and intermediate state clusters are associated with definitions that comprise a cluster definition that each instance of a state is determined to be a member of (or not). A starting point cluster definition can be associated with a particular disease, for example a particular type of cancer. During training and retraining, the trained deep neural network(s) determine associated intermediate states, state clusters, state transition trajectories, and edges that can be used to predict a particular endpoint for that disease, for example a clinical endpoint including a cure, remission or death.

### Individual State Trajectories and Prognosis Generation

In addition to probabilistic prognosis graphs (PPG1, PPG2), FIG. 20A also illustrates multiple individual patient trajectories corresponding to patients (Pt. 1-4). Each individual patient trajectory includes a starting state (SS1, SS2, SS3, SS4) which includes a clinical starting state. A clinical starting state includes initial history information, the date stamp for the starting state, and a diagnosis of a disease or condition provided by a health care provider (HCP), for example, diagnosis of a particular cancer, neurological, or other disorder. Alternative starting states include a clinical state determined by the trained machine learning algorithm and/or trained deep neural network, upon collection of measurement data that is not associated with a diagnosis provided by an HCP, for example measurement data collected when a user first enrolls in a longitudinal tracking clinical study. Each of the starting states comprises a starting point cluster (SPC) according to an associated starting point cluster definition. A starting profile cluster is defined as a group of starting profiles that have similar characteristics. Assignment of a starting profile to a particular cluster will typically be based on a diagnosis determined by a medical professional. In this arrangement, multiple starting profiles are included in a cluster because they are all from patients with the same diagnosis, for example, a first cluster might include profiles from patients having pancreatic cancer and a second cluster might include profiles from patients having colon cancer.

Each individual patient trajectory includes one or more immune or clinical states (e.g., state 1-1, state 1-2) which are calculated by POPS (2000) based on collected and augmented data. POPS 2000 further calculates trajectories between the states, for example vector 1[1,2] between states (1-1) and (2-2). These calculated intermediate states include, at each intermediate time point, a patient profile that includes values of collected data, for example, PBMC population and abundance at the time point, and which can include other values, for example laboratory test result values such as hematocrit and albumin, images from an MRI scan, or microbiome characterization based on bacterial nucleic acid sequences. In an exemplary embodiment, the machine learning/clustering program (e.g., 1320, 2320) determines a first state (e.g., state 1-1) and a trajectory from the starting state (SS1) to the first state.

The POPS (2000) matches an individual trajectory representation against a particular graph representation. The POPS (2000) determines matches between the calculated states and stored state or node definitions to categorize the individual's current state as a member of a particular state cluster represented in the graph representation and determines matches between calculated trajectories between states and edge definitions to categorize a particular calculated trajectory between states as comprising a particular edge. In this manner, for a particular individual trajectory, the POPS (2000) determines a corresponding location in the probabilistic prognosis graph.

In one arrangement, the machine learning/clustering program determines membership of the first state in a state cluster by determining that a set of characteristics including, for example, patient profile, previous state, and trajectory to the first state, falls within a particular intermediate state cluster definition (e.g., definition of state cluster 1-1) and corresponding edge definition (e.g., definition of edge (1-1)). In this manner, an intermediate state corresponds to a cluster of profiles with similar characteristics and with similar trajectories to the intermediate state. Non-limiting exemplary intermediate states include: responding to treatment; not responding to treatment; stable disease; disease progression; etc.

A trajectory between states is defined as a vector that encodes changes in one or more profile values (e.g., changes in T cell count from a previous time point (i−1) to a current time point (i)) that includes changes in magnitude, direction of change, and velocity of change. For example, a first trajectory between a first pair of profiles, each measured at a different time point, could include a drop in T cell count (negative direction and/or magnitude) over a time period of a week while a second trajectory between a second pair of profiles could include an increase in T cell count (positive direction and/or magnitude) over a month. In practice, a profile can include multiple different measured parameters and a trajectory between time points can be represented as a tensor comprising multiple vectors, scalars, and/or additional tensors.

In a non-limiting exemplary implementation, the prognosis estimator program (2410) processes collected data corresponding to patient 1 starting state (SS1) and first state (1-1) and calculates a trajectory (vector 1[S,1]) between the states. The prognosis estimator program determines mapping between the states and trajectory to starting point cluster (SPC), state cluster (1-1), and edge (1-1) and, based on the mapping, determines that patient 1 is at node state cluster (1-1) of probabilistic prognosis graph (PPG1). The prognosis estimator program then traverses the probabilistic prognosis graph (PPG1) along edges (1-2) and (1-3) through state (1-2) and to a specific endpoint cluster (e.g., EPCA).

The prognosis estimator program generates output that includes a current state (defined by state cluster 1-1 definition), a prognosis (i.e., a clinical endpoint state represented by an endpoint cluster (EPCA)), transition events between states, e.g., a treatment associated with edge (1-2), and a probability calculated based on probabilities associated with transitions between states of the probabilistic prognosis graph (PPG1). If the individual trajectory for patient 1 is updated, e.g., when the POPS (2000) receives immune data corresponding to state 1-2, the prognosis updater program (2420) runs the prognosis estimator program to generate a new prognosis and compares the new prognosis to the old prognosis.

A definition of a clinical endpoint cluster includes a resolution of a clinical state, for example a diagnosis that a patient is free of cancer or in remission, or that a patient has died. In alternative arrangements, an endpoint can include a state that is not associated with a particular diagnosis or disease, for example a state at the end of a monitoring trial.

The machine learning algorithm and/or deep neural network predicts, based on the trajectory, that patient 1 will reach a specific endpoint (e.g., A). When data from a second longitudinal time point becomes available, the machine learning algorithm and/or deep neural network processes the data to determine a state corresponding to that second longitudinal time point (e.g., state 1-2) and a trajectory that extends from the initial state to state 1-1 and from there to state 1-2. The machine learning algorithm and/or deep neural network determines, based on the trajectory, that the predicted endpoint for patient 1 continues to be a specific endpoint (e.g., an endpoint associated with EPCA). When patient 1 reaches an endpoint, the machine learning algorithm and/or deep neural network is retrained on patient 1 data to update algorithm weights.

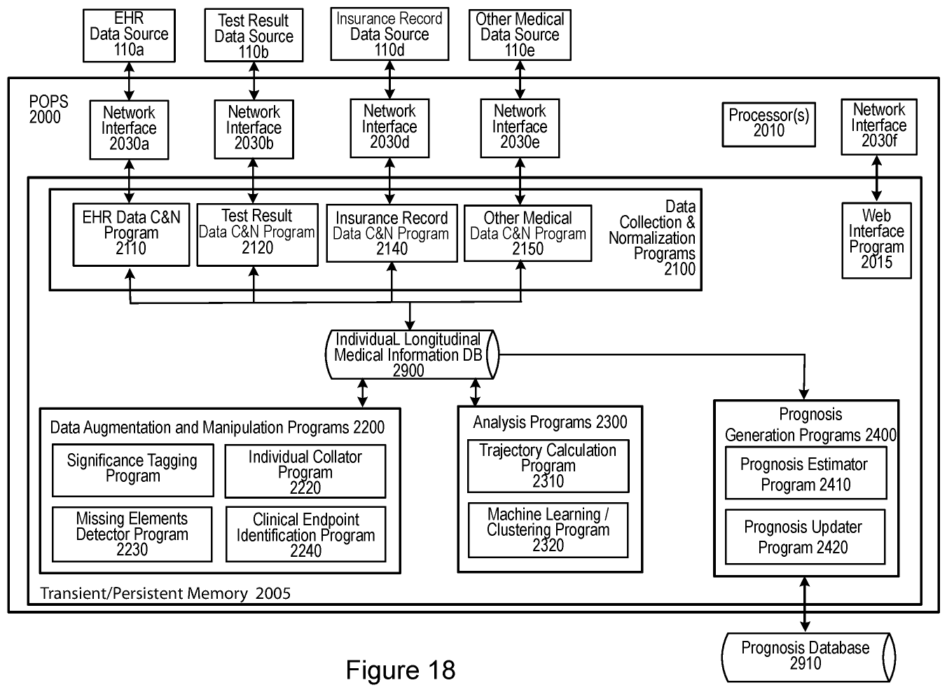

Referring to FIGS. 17 and 18, the machine learning/clustering program and/or deep neural network (e.g., 1320, 2320) can be trained to recognize profile characteristics that are most closely associated with or that are most diagnostic for each of multiple assigned starting profile clusters. In this arrangement, a profile corresponding to a new patient may be assigned to a starting profile cluster by the trained machine algorithm.

Referring to FIG. 20A, the PPNGGS (1000) and/or POPS (2000) determines, or is informed by an operator, that multiple starting profiles are part of a starting profile cluster, and the corresponding starting profile cluster definition. Similarly, the system determines the endpoint/clinical outcome profile cluster definitions. An endpoint/clinical outcome profile cluster defines a boundary region such that individual profiles that lie within the boundary region are considered to correspond to the endpoint indicated by the cluster. In some arrangements, an ending profile cluster is defined as a group of endpoint profiles that each correspond to a state determined externally to the machine learning algorithm and/or deep neural network, for example by a clinician. Exemplary endpoints include death, acute remission, acute regression, in some cases followed by remission, acute progression, and death.

The machine learning/clustering program and/or deep neural network (e.g., 1320, 2320) can be trained to recognize profile characteristics that are most closely associated with or that are most diagnostic for each of multiple assigned endpoint profile clusters. These characteristics include patient profile parameters corresponding to the assigned endpoint and can also include one or more states that precede the endpoint and a trajectory that terminates at the endpoint. In this arrangement, a profile corresponding to a new patient may be assigned to an endpoint profile cluster by the trained machine algorithm and/or deep neural network.

The system can collect additional longitudinal measurements for time points following an endpoint cluster time point. For example, after a patient has been declared to be cancer free or in remission, follow-up tracking of profile data can be performed to monitor the ongoing state of the patient. In some cases, a new endpoint may be reached by the patient. For example, a cancer in remission may become active again in the patient or a patient who was declared cancer free may receive a new, and in some cases different, cancer diagnosis. The machine learning algorithm and/or deep neural network is trained on follow-up tracking data and any endpoints that are reached. Based on this training, the machine learning algorithm and/or deep neural network can determine new endpoints, for example cancer free but likely to develop other cancers or in remission and likely to remain in remission.

Individual trajectory Outside of Variance of PPG

The PPNGGS (1000) determines a graph variance which indicates upper and lower limits of trajectories between a starting state and an endpoint along a trajectory, and encodes this variance as part of the probabilistic prediction graph representation. Referring to FIG. 20B, the POPS (2000) can detect when a trajectory of states for a particular patient begins to trend outside of the graph variance, or if too many patients follow a low probability graph path, the system initiates retraining of the deep neural network(s) to include the new patient trajectory data. In this case, a trajectory corresponding to patient 5 includes a first state (state 5-1) and a second state (state 5-2) that both are contained within the variance of edge (1-2) from the starting point cluster (SPC) to an endpoint cluster (e.g., EPCA). The trajectory includes a third state (state 5-3) that falls outside of the illustrated variance. When an endpoint of patient 5 is reached, that patient's trajectory is identified as being out of the defined variances for the graph. The patient's data and trajectory information are provided to the PPNGGS (1000) for use in subsequent retraining of the machine learning algorithm. Retraining may be immediate, or batch, so that at some point in the future, the machine learning algorithm is retrained to include data of patient 5. In either case, after retraining, the graph representation is regenerated and the updated graph representation is made available for use in subsequent predictions. In this way, an iterative learning feedback system is created that allows the system to iteratively learn as it encounters new information. If the trajectory falls within the specific endpoint cluster (e.g., EPCA), the machine learning algorithm and/or deep neural network may determine an updated variance for a probabilistic prognosis graph between the starting state and a specific endpoint cluster. If the endpoint associated with patient 5 does not fall within the endpoint cluster, the machine learning algorithm and/or deep neural network may generate a new probabilistic prognosis graph, or update an existing probabilistic prognosis graph, between the Starting Profile Cluster and the patient 5 endpoint or an End Cluster that the patient 5 endpoint falls within.

### Individual Immune States, Trajectories, and Disease States

### Individual Immune Trajectories

Individual immune trajectories are computed from available multiomics data on a patient-by-patient basis. They may be complete (e.g., because the patient is no longer undergoing treatment) but often they will be for a patient who is beginning treatment or is still undergoing treatment. Individual longitudinal immune trajectories are computed based upon longitudinal patient data (i.e., data about the patient captured at different times) and combined into a consolidated longitudinal immune trajectory incorporating generalized disease states, immune states, and clinical outcomes and their characterizations. The consolidated longitudinal immune trajectory can be used to determine range values for similar immune/clinical states as the levels of expression and amount of time required to achieve desired responses vary by patient. Such longitudinal immune trajectories are typically updated from time to time as patients progress in their treatment. Furthermore, some embodiments use group immune trajectories (data from plural or many patients) as opposed to data from a single patient.

The exemplary system further enriches prediction and annotations as described above in connection with FIGS. 6A & 11 by using advanced analyses to add significant data that was not present in the original input data set. Particular enrichment techniques as described above increase the accuracy and timeliness of predictions such as, for example, whether the patient is likely to attain a clinical endpoint such as survival since if an immune-based therapy is not having a desired immunological impact, the patient is unlikely to attain the desired clinical endpoint. Furthermore, the variance in the immunological impact on the consolidated immune trajectory is an indication that a therapy is succeeding or failing earlier in the course of treatment.

The exemplary system identifies insights from the database of longitudinal measurements of immune sequencing data associated with the clinical data (such as treatment codes, record timestamps) in response to clinical actions, including the actions, response, and resistance to various therapies, in order to identify a set of longitudinal profiles of immune responses measured or observed in patients over time.

For example, cancer patients treated with checkpoint-blockade therapies may still have a large tumor after therapy. It is difficult to differentiate between cases in which the patient had no immune response to the drug (which is a therapy failure), cases in which the patient did have a measurable immune response that wasn't enough to reduce or eradicate the tumor (a partial response), cases in which the patient had an immune response sufficient to eradicate the cancer, and various other cases for which future treatments and prognoses differ. The first case (no response) follows a different immune trajectory from the partial and complete response cases (partially successful and completely successful therapies respectively) in which the partial success case may take additional time because of a reduced immune response.

Longitudinal immune trajectories each may integrate clinical diagnosis, treatment, and immune state information over time in a manner that permits similar trajectories to be generalized. Longitudinal immune trajectories each thus may describe sets of immune state/treatment options that produce similar outcomes. For example, a first patient with a high immune response and a second patient with a medium immune response may follow the same immune trajectory differing only, for example, by the levels of various proteins and antibodies expressed in response to a treatment, and/or the length of time it takes for the transition from a first immune state to a second immune state to occur. This allows the exemplary system to calculate the probability of a patient's following a first trajectory or a second trajectory on the combined basis of their current (and past) immune state(s) and current diagnosis/treatments matching the definitions of each of the longitudinal immune states while allowing for differences in individual patients' immune states.

FIG. 20C illustrates exemplary relationships between immune states at each of multiple time points, and clinical states corresponding to the immune states, individual trajectories between states, and time points. An immune state at a particular time point (e.g., starting state SS7, immune state 7-1) includes measurement values related to immune system parameters generated or gathered at that time point and information about the immune system parameters (cell types, cell states, etc.) generated using values of the longitudinal measurements. The measurements include, for example, single cell RNAseq (scRNAseq) and CITE-seq data from peripheral blood mononuclear cells (PBMCs), the T receptor information as described above, other multiomics data, and other cells, imaging data, microbiome characterization, proteome characterization, and others. In this manner, an immune state represents a snapshot of measurable aspects of a patient's physiology, microbiome, etc. at a particular point in time.

A predicted endpoint state—at a particular time point is a function of the patient's immune state at the particular time point, the patient's disease and immune state at one or more previous time points, and a trajectory of immune states, or components of immune states, that includes information from the patient's immune state at the particular time point and information from the patient's immune state at one or more previous time points. There are many other factors that affect an individual's immune state; for example, the previous paragraph mentions other measurements, including characterization of the microbiome (presumably gut microbiome, not skin or other microbiome), proteome characterization, etc. So a predicted endpoint state is not necessarily just a function of the patient's immune state. For example, suppose that a patient has been infected with HIV, and so is immune-suppressed. In that case, the immune state is relevant, but so is the HIV status.

In a non-limiting exemplary implementation shown in FIG. 20C, two patients have the same or similar measured immune states at a particular time point, but their disease states at that particular time point are not the same. Each patient's disease state at the particular time point is a function of a trajectory to that time point from a previous time point, so if trajectories that lead to the immune state are not the same for both patients, corresponding disease states of each patient may not be the same.

Referring to FIG. 20C, individual immune state measurements and immune state trajectories corresponding to two patients (Pt. 6 and Pt. 7) are illustrated. Patient immune states at intermediate time points and at endpoint time points are illustrated. At each of multiple intermediate time points, an immune state and disease state are associated with each patient. In the simplified example shown in FIG. 20C, an immune state is defined as a T cell count at a particular time point, which may be determined based on scRNAseq data but in embodiments herein will typically be based on multiomics. Although the immune states illustrated in FIG. 20C include a single variable for ease of understanding, it should be understood that a typical immune state includes multiple variables (e.g., presence and abundance of multiple cell types determined by scRNAseq or DNAseq, possibly microbiome constituents, test result parameter values (e.g., hematocrit, protein levels, etc.), imaging results, and additional information from EHRs). In one arrangement, each of the multiple immune state parameters is represented as one coordinate of a plurality of coordinates that define a profile in an N-dimensional space. It should be noted that some may argue that microbiome does not constitute part of a patient's “immune state”—i.e., certain microbiome profiles can be used to predict likelihood of benefit from checkpoint inhibitor treatment, but depending on the embodiment, it may or may not reflect changes in the patient's immune system.

Intermediate time points are joined by edges that represent trajectories between time points. Each trajectory can be defined as a vector of one or more profile measurement values between time points. In the illustrated example, a vector represents a change in T cell count between time points. A disease state at each time point (i) is function of both the immune state at the time point (i) and a vector from an immune state at a preceding time point (i−1) to the immune state at time point (i). In some arrangements, a disease state at time point (i) is a function of trajectories between immune states at multiple preceding time points.

Referring to patient 6's trajectory, an immune state (6-3) for patient 6 that corresponds to a disease state (6-3) includes a particular T cell count or T cell count range. An immune state (7-3) for patient 7 that corresponds to disease state (7-3) has essentially the same T cell count as in immune state (6-3). In other words, the immune states of patients 6 and 7 appear to be the same at a time point that corresponds to both immune state (6-3) and immune state (7-3). However, despite the immune states being similar, a corresponding disease state of patient 6 (disease state 6-3) is not necessarily the same as a corresponding disease state of patient 7 (disease state 7-3) because the disease states are each a function of both the corresponding immune state and the trajectory or vector that leads to the immune state.

A trajectory for patient 6 leads to immune state (6-3) (which corresponds to disease state 6-3) from immune state 6-2 (corresponding to disease state 6-2), which includes a higher T cell count than the T cell count corresponding to immune state (6-3) (e.g., 4000). Thus, a vector from immune state (6-2) to immune state (6-3) (vector 6[2, 3]) includes a decrease in T cell count with a negative direction.

In contrast, a trajectory for patient 7 leads to immune state (7-3) (corresponding to disease state 7-3) from immune state (7-2) (corresponding to disease state (7-2)), wherein immune state (7-2) includes a lower T cell count than the T cell count of immune state (7-3). A vector from immune state (7-2) to immune state (7-3) (vector 7[2, 3]) thus includes an increase in T cell count with a positive direction. Assuming that increasing T cell count is indicative of an improvement in disease state (e.g., that an increased number of T cells indicates that a patient's immune system is successfully gearing up to fight a cancer), POPS (2000) may tag disease state (7-3) as “regression” and disease state (6-3) as “acute progression.”

The POPS (2000) predicts different immune states, different disease states, and clinical endpoints or outcomes for patients 6 and 7 following immune states (6-3) and (7-3) based on trajectories or vectors leading to these immune states. As illustrated in FIG. 20C, the POPS processes individual trajectory measurement data that includes immune states (6-3) and (7-3) and generates a prediction that patient 6 will reach an endpoint state that is part of endpoint cluster B (EPCB) and a prediction that patient 7 will reach an endpoint state based on an endpoint cluster type A definition. In an exemplary arrangement, the POPS tags state A as “remission” and state B as “death.”

### Exemplary Training/Learning of the Machine Learning Algorithm

In an exemplary arrangement, the PPNGGS (1000) employs a recursive learning method wherein the PPNGGS begins from a known state and attempts to learn aspects of unknown states that are related to the known state. An exemplary known state is a state, or group of states, that fall within an endpoint cluster or that otherwise corresponds to a known endpoint state such as a clinical diagnosis of remission, acute progression, or death. The PPNGSS determines one or more preceding states with unknown characteristics (i.e., uncharacterized states that preceded the known state) and attempts to learn characteristics of the preceding states that are predictive of transitioning from the preceding state to the known endpoint state.

For example, the PPNGGS (1000) determines that longitudinal measurements corresponding to a group of patients whose trajectories reached a first known endpoint/clinical outcome state (e.g., a remission state) include a first cluster of similar profiles determined for a time point that preceded the first known state (i.e., a cluster of similar preceding profiles). The PPNGGS (1000) further determines that a second cluster, different from the first cluster, of similar preceding immune states includes immune states corresponding to patients whose trajectories did not reach the first known endpoint state or whose trajectories reached a second, different, known endpoint state (e.g., patients who reached a progression state rather than a remission state). The PPNGGS 1000 then determines differences between (preceding) first and second clusters of immune states that are predictive of reaching the first endpoint state, not reaching the first endpoint state, or reaching the second endpoint state.

In an exemplary arrangement, the PPNGGS (1000) determines, for each preceding immune state, a preceding state that is a function of the preceding immune state and of a trajectory of one or more immune state features that lead to the preceding profile. This concept is illustrated in FIG. 20C. The PPNGGS (1000) determines that one of the similar immune states (6-3) preceded endpoint state B while another of the similar immune states (7-3) preceded endpoint state A. The PPNGGS (1000) determines that a trajectory to immune state (6-3) included vector 6[2,3] which is characterized by a drop in T cell count. The machine learning model determines that an immune state (e.g., 6-3) with a preceding trajectory that includes a drop in T cell count (e.g., vector 6[2,3]) corresponds to an immune or disease state that is predicted to proceed to outcome B (e.g., progression). The machine learning model and/or deep neural network determines that an immune state (e.g., 7-3) with a preceding vector that includes an increase in T cell count (e.g., vector 7[2,3]) corresponds to an immune or disease state that is predicted to proceed to outcome A (e.g., regression).

The PPNGGS (1000) assigns weights to features that comprise the disease states (immune profiles and vectors, or individual constituents of each) and that are more or less predictive of proceeding to a particular immune or disease state. The PPNGGS steps through additional immune states or clinical endpoints with unknown characteristics, learning predictive aspects and assigning weights, until it reaches another known state such as a cluster of starting profiles. In this manner, the PPNGGS (1000) determines trajectories between known disease and immune states that can be used to predict future states and outcomes based on newly collected patient information. In one embodiment, such weights can be assigned as neural network level coefficients through training of one or more deep neural networks.

### Exemplary Machine Learning Algorithms

In an exemplary arrangement, referring to FIGS. 17 and 18, a machine learning/cluster program (e.g., 1320, 2320) includes an immune state profile algorithm, a disease state prediction algorithm, and endpoint prediction algorithm. Machine learning/cluster programs may include deep neural network and related computing architectures, including one or more of the following: convolutional neural networks (CNNs), recurrent neural networks (RNNs), reinforcement learning (RL), inverse reinforcement learning (IRL), deep reinforcement learning (DRL), generative adversarial networks (GAN, and GAN-like variants, including adversarial auto-encoders which include variational auto-encoders (VAE) described above).

The immune state profile algorithm generates a patient immune state at each time point based on the individual measurements (e.g., single cell genomic and/or transcriptome measurements) at each time point. A patient immune state includes a defined subset of potentially available data including measured parameters or parameters inferred from measured parameters (e.g., T cell populations and abundances based on the scRNAseq data, identification of bacteria comprising the gut microbiome based on bacterial sequence data) and other data including imaging data at the point in time. The subset of potentially measurable data that comprises a patient immune state is learned by the machine learning/cluster program and may change during re-training of the algorithm as data from additional patients is added.

In some arrangements, an immune state profile algorithm selects an initial immune state profile that includes a specific group of profile components, e.g., diagnostic biomarkers (including abundance of particular T cell populations and activation states, proteins, metabolites, etc.) that should be generated and provides the selected profile to the profile algorithm. A selected profile is a function of initial disease state and treatment (e.g., particular biomarkers to track pancreatic cancer).

In one arrangement, the immune state profile algorithm determines, based on processing training data, that a particular set of parameters are useful for clustering analysis and that changes in the parameters are predictive of a trend toward one of a first outcome and a second outcome. Based on these determinations, the immune state profile algorithm includes the particular set of parameters in an immune state profile. The immune state profile design algorithm may also assign weights to some or all of the parameters in an immune state profile where the weights indicate the relative diagnostic importance of the parameters. While processing data from new patients, the immune state profile algorithm is re-trained and may adjust which parameters are included in an immune state profile and may adjust weights associated with the included parameters based, for example, on differences between predicted and observed outcomes and/or intermediate states.

The disease state prediction algorithm predicts a disease state at a point in time based on one or more of: trajectory of immune states (and/or one or more immune state components); current immune state; and one or more past disease states.

The endpoint prediction algorithm generates, at a particular time point, a prediction of a clinical endpoint for a patient based on the immune state at the particular time point and a trajectory of immune and/or disease states leading to the particular time point, as discussed previously.

Each of the immune state profile algorithm, disease state algorithm, and end point prediction algorithm include weights that are updated at each iteration of the method (i.e., from P0 to Pn), for example based on comparison of disease state predicted by the machine learning/cluster program and observed disease state of a patient (at one or more Pi and/or Pn) and/or comparison of an endpoint predicted for a patient and observed state.

If the disease state prediction algorithm cannot determine a predicted disease state with greater than a threshold level of confidence, the immune state profile algorithm can determine a new or modified immune state profile. The system may perform additional sample processing to collect or request data needed for a new or updated immune state profile (e.g., targeted PCR to characterize a biomarker that was not characterized on first pass). The immune state profile algorithm reprocesses data from the current time point to generate the new or updated immune state profile, which the disease state algorithm processes to determine a disease state. These steps may be iterated until a disease state is determined with confidence greater than the threshold value. The new or modified immune state profile may be used at subsequent time points. In some arrangements, the new or modified immune state profile is used to retrain the machine learning/cluster program.

The systems and methods described herein further provide a mechanism for collecting health information from diverse sources and processing it to calculate and produce ongoing probabilistic predictions regarding a patient's health and treatment outcome(s) over time, where the predictions are based at least in part upon the patient's immune and clinical states (collectively, the disease state). The described exemplary systems and methods of operation provide improved data collection, including dynamically updated data sets that integrate additional collected data as it becomes available, augmentation of collected data and enhancement in order to complete missing, incomplete, and/or contradictory collected data, improved computational performance, separation of training and prediction data, improvements in cell type classification resulting in more accurate predictions regarding a patient's diagnosis and treatment, and for feedback to the model training process when patient-specific predictions deviate from the known probabilistic prognosis graph.

Not all diseases have immune state characterizations that permit their integration into immune trajectories; the system extends known immune state characteristics in order to address these additional disease conditions in a process called transferability. The exemplary system transfers knowledge learned in classical immune settings, such as infection or cancer immunotherapy, to other diseases in which the immune system plays a critical role by using transfer learning based upon the commonality of immune states/snapshots. In an embodiment, the system identifies the functional molecules (proteins, antibodies, T cell receptors) expressed by each cell type, and how these same functional molecules vary in healthy individuals, in the setting of disease, and in response to therapy. Such proteins may includes any number of proteins: immune cell specific markers (e.g., CD4, CD8), HLA type, cytokine secretion profiles, other immune cell-specific surface protein markers (e.g., CD45RA, etc.), TCR alpha/beta, TCR gamma/delta, etc., but also the epigenetic regulators and other things that don't necessarily reflect a specific immune state (at least directly) as mentioned above. In one embodiment, the functional molecules might comprise immune effectors and immune-related surface proteins, but epigenetic regulators, histones, transcription factors (etc.) could all be classified as “functional” molecules in immune cells even if they don't directly reflect a specific “immune state” in the same way CD4 or CD8 might be thought of doing so. The described system generalizes these attributes and synthesizes the learnings from the immune state data and generalizes those immune attributes to enable an end user to diagnose and treat additional disease types and to monitor the impact of treatment decisions on disease etiology and progression, in order to support numerous additional disease types and diagnosis codes by the system, and to identify and differentiate mechanisms of action for various therapies and their associated immune state changes.

In one embodiment, the described computing system calculates a probabilistic prognosis graph based upon collected and processed longitudinal clinical health data, integrated into multiple clinical and immune snapshots collected over time, general genomic information collected from research papers and related studies, and additional information related to immune responses associated with specific treatments; automatically processes this collected information in order to calculate values and parameters for one or more longitudinal immune trajectories, and then combines these trajectories into a probabilistic prognosis graph. The resulting graph representation is then exported to a privacy preserving (i.e., HIPAA-compatible) prognosis generation system, which uses the generated graph representation to generate individualized probability-based prognosis sets using an individual patient's immune state and/or immune trajectory, and then tracks the patient's evolving clinical medical data and immune snapshots in order to update the patient's immune state/trajectory and the resulting probability-based prognosis(es) in accordance with the newly collected information. Related methods of operating the described computing apparatus are also disclosed.

The system leverages immune state-transferability, longitudinal and individualized immune trajectory definitions, advanced machine learning, and privacy-preserving probabilistic graph techniques to provide these benefits.

### Immune State-Transferability

The computing system and its methods of operating provide a multiomic profiling solution for individual immune cells found in blood or tissue. Multiomic refers to the integration of multiple data sets derived from characterization of various analytes, such as the genome (i.e., genomic sequence data), proteome (i.e., protein expression data), transcriptome (i.e., gene expression data), epigenome (i.e., genome and/or histone modification data), metabolome (i.e., metabolite data), and microbiome (i.e., a meta-genome and/or meta-transcriptome, depending upon how it is sequenced). An exemplary profiling solution for individual immune cells found in blood or tissue incorporates all available data as inputs, and produces immune trajectories and prognosis graphs as output. This allows the system to use the relevant data about each sample (such as methods of data collection that produce known inaccuracies (i.e., batch effects) or missing data (i.e., dropouts in scRNAseq), and variations in processing samples), about individual cells (such as their cell type, if known beforehand), as well as relevant clinical data (disease type and stage, medical treatment history, clinical response data, etc.).

An integrative analysis of these features together with the patient's immune cell type and gene expression profile at each stage produces a full view of an individual's immune state, and when combined with clinical information, including disease prognostic factors (e.g., age, smoking, BMI), can be used to precisely understand health and disease states. The collection of this knowledge for an individual is sometimes referred to as an “individual immune trajectory”, which describes the individual's disease and immune states over time coupled with clinical information, demographic, and disease prognostic factors.

### Exemplary Machine Learning Techniques

The exemplary system integrates the collected input data using one or more deep neural network (“DNN”) techniques, including for example single pass-variational autoencoder (VAE), for dimension reduction and auto-tagging in order to create a unique dataset comprising collected information, one or more longitudinal immune trajectories, and a probabilistic prognosis graph representation that is used for ongoing clinical predictions. In one exemplary embodiment, collected information incorporated into the neural networks includes:
Immune monitoring data, collected across multiple indications (tumor-agnostic) and disease types.
Immune cell type frequency data from a wide variety of conditions, associated with pre-training weights.
Longitudinal clinical histories for a statistically valid sample of patients, including information collected at multiple time-points for each patient (samples taken prior, during, and after treatment) and assembled into a set of patient-specific clinical trajectories, each appropriately tagged with clinical milestones and endpoints.
Metadata, including clinical data (disease indication, stage, medical treatment history, response data), imaging data (or parameters taken from it), or other disease-centric data (e.g., tumor mutational burden (TMB), microsatellite instability (MSI), expression levels of immune checkpoint proteins, such as PD1 or PDL-1, human leucocyte antigen (HLA) expression data, and the like).

The exemplary system achieves additional computational efficiencies by utilizing a VAE (or other, e.g., deep neural network multitask) machine learning architecture in order to reduce the dimensionality of the collected data by eliminating input data dimensions in the VAE outputs. This simplifies the immune state definitions, which allows the identification of commonalities that characterize specific immune trajectories.

Machine learning neural network (e.g., VAE) components of the exemplary system implement a Multi-task Transfer Learning approach to provide batch harmonization (i.e., adjusting for batch effects associated with individual datasets), joint dimensionality reduction, lower dimension visualization, cell type identification, an automated way to annotate cells, and a multi-task learning framework capable of identifying and transferring immune markers across disease indications and treatments, as well as identifying disease-specific markers for studying mechanisms of action and resistance.

The exemplary system further improves computational efficiency by training one or more neural networks to learn patterns from multiple diseases and treatments within the same or different network(s). This multi-task learning technique enables transfer of immunological patterns across disease indications and treatments by identifying those diseases, treatments, and responses that share certain patterns of immunological response, enabling the system to predict disease trajectories and clinical response for a broad range of treatment protocols.

The exemplary system further incorporates into the neural network components, weights trained on cell populations which are found only in particular patient subsets and transfers those weights into the broader set of input data types as they are processed. This approach, when integrated with VAEs, allows the system to differentiate cell types that it would otherwise not be possible to distinguish. This enables the improved cell type identification illustrated in FIG. 1B to be completed, providing superior cell type assignment and/or rare cell type identification.

Thus, by applying deep neural networks (e.g., trained VAEs) specifically tailored to immunology, the commonality of certain immunological features can be utilized to transfer knowledge learned in cancer immunotherapy to enable diagnostic and disease monitoring models for many other immune-heavy settings (autoimmune disease, cardiovascular disease, neurodegenerative disease, and many other disorders which derive from common pathways of immune dysfunction).

### Privacy Preserving

The exemplary system provides privacy preserving attributes in that the training and predictive aspects of the system are separable. This enables the prediction system to require no access to the neural network generation datasets (which generally comprise a large set of patient-specific longitudinal information that will need to be protected or isolated under various national privacy laws or regulations), and provides for the one-way transfer of the probabilistic prognosis graph representation.

All patents and publications cited above herein are expressly incorporated by reference for purposes of enablement and written description, but not for purposes of any potential disclaimer of subject matter.

It will also be recognized by those skilled in the art that, while the technology has been described above in terms of preferred embodiments, it is not limited thereto. Various features and aspects of the above-described technology may be used individually or jointly. Additionally, features employed in any embodiment described above may be used individually or in combination with features of any other embodiment. Further, although the technology has been described in the context of its implementation in a particular environment, and for particular applications, those skilled in the art will recognize that its usefulness is not limited thereto and that the present technology can be beneficially utilized in any number of environments and implementations. Accordingly, the claims set forth below should be construed in view of the full breadth and spirit of the technology as disclosed herein.

What is claimed:

1. A method automatically performed with at least one processor, comprising:
receiving clinical data that was collected over time;
based on the received clinical data that was collected over time, generating graph-based longitudinal trajectories indicating predicted outcome and/or treatment or exposure effectiveness for at least one disease;
receiving multiomic immune state data measured for at least one patient having said disease, the multiomic immune state data including at least RNA marker data, T cell receptor marker data and B cell receptor marker data for a plurality of different immune system single cell populations;
mapping the graph-based longitudinal trajectories to the multiomic immune state data; and
based on the mapping, isolating at least one distinct subset of the plurality of different immune system single cell populations the mapping indicates are exhibiting evolving molecular changes.

2. The method of claim 1 wherein isolating includes developing RNA/protein heatmaps per cell subset.

3. The method of claim 1 wherein the mapping includes automated cell type prediction.

4. The method of claim 3 wherein the mapping includes reducing dimensionality, extracting features, correcting for multiomic batch effect and removing multiomic based multiplets.

5. The method of claim 1 wherein the isolating includes separating sub-cell types.

6. The method of claim 1 wherein the mapping includes cell type-specific matching of clinical signatures with perturbation signatures, including mapping signatures against large scale CRISPR perturbations.

7. The method of claim 1 wherein mapping includes signature mapping to clinical covariates.

8. The method of claim 7 further including determining association of complex molecular phenotypes with clinical covariates.

9. The method of claim 1 further including validating annotation to group clones of a specific cell type/cell type groups by their trajectories over time.

10. The method of claim 8 further including enriching trajectories for response and/or treatment clinical covariates.

11. The method of claim 9 further including enriching trajectories in specific molecular phenotypes of interest.

12. A system for automatically detecting evolving immune system molecular changes, the system including:
at least one processor and an output, and
a memory connected to the at least one processor, the memory storing:
instructions,
clinical data that was collected over time, the clinical data including multiomic immune state data measured for at least one patient having a disease, the multiomic immune state data including at least RNA marker data, T cell receptor marker data and B cell receptor marker data for a plurality of different immune system single cell populations;
the at least one processor upon executing the instructions being configured to perform operations comprising:
based on the received clinical data that was collected over time, generating graph-based longitudinal trajectories indicating predicted outcome and/or treatment or exposure effectiveness for the at least one disease,
mapping the graph-based longitudinal trajectories to the multiomic immune state data, and
based on the mapping, isolating at least one distinct subset of the plurality of different immune system single cell populations the mapping indicates are exhibiting evolving molecular changes.

13. The system of claim 1 wherein isolating includes developing RNA/protein heatmaps per cell subset.

14. The system of claim 1 wherein the mapping includes automated cell type prediction.

15. The system of claim 14 wherein the mapping includes reducing dimensionality, extracting features, correcting for multiomic batch effect and removing multiomic based multiplets.

16. The system of claim 1 wherein the isolating includes separating sub-cell types.

17. The system of claim 1 wherein the mapping includes cell type-specific matching of clinical signatures with perturbation signatures, including mapping signatures against large scale CRISPR perturbations.

18. The system of claim 1 wherein mapping includes signature mapping to clinical covariates.

19. The system of claim 18 further including determining association of complex molecular phenotypes with clinical covariates.

20. The system of claim 1 further including validating annotation to group clones of a specific cell type/cell type groups by their trajectories over time.

21. The system of claim 20 further including enriching trajectories for response and/or treatment clinical covariates.

22. The system of claim 21 further including enriching trajectories in specific molecular phenotypes of interest.
